SEQUENCE LISTING (2 sequences)

SEQ ID NO: 1 - protein, 390 aa, Homo sapiens
MRGDRGRGRGGRFGSRGGPGGGFRPFVPHIPFDFYLCEMAFPRVKPAPDETSFSEALLKRNQDLAPNSAEQASILSLVTKINNVIDNLIVAPGTFEVQIEEVRQVGSYKKGTMTTGHNVADLVVILKILPTLEAVAALGNKVVESLRAQDPSEVLTMLTNETGFEISSSDATVKILITTVPPNLRKLDPELHLDIKVLQSALAAIRHARWFEENASQSTVKVLIRLLKDLRIRFPGFEPLTPWILDLLGHYAVMNNPTRQPLALNVAYRRCLQILAAGLFLPGSVGITDPCESGNFRVHTVMTLEQQDMVCYTAQTLVRILSHGGFRKILGQEGDASYLASEISTWDGVIVTPSEKAYEKPPEKKEGEEEEENTEEPPQGEEEESMETQE

SEQ ID NO: 2 - protein, 8 aa, Homo sapiens
EESMETQE

(19) United States
(12) Patent Application Publication
(10) Pub. No.: US 2012/0021432 A1
(43) Pub. Date: Jan. 26, 2012

(54) Phosphorylated NF45 Biomarkers, Antibodies And Methods Of Using Same

(75) Inventors: Yi Yu, Malden, MA (US); Xiubin Gu, Wilmington, MA (US); Xiaolan Zhao, Andover, MA (US); Carol Waghorne, Lexington, MA (US); Thomas C.K. Chan, Winchester, MA (US); Taiping Chen, Andover, MA (US)

(73) Assignee: ArQule, Inc., Woburn, MA (US)

(21) Appl. No.: 13/087,457

(22) Filed: Apr. 15, 2011

Related U.S. Application Data
(60) Provisional application No. 61/324,964, filed on Apr. 16, 2010.

(51) Int. Cl. G01N 33/566; C07K 16/18; C07K 14/435; G01N 21/64

(52) U.S. Cl. 435/7.1; 436/501; 530/387.9; 530/352

(57) ABSTRACT

The present invention relates to isolated phosphorylated NF45 peptides and isolated phosphorylation site-specific antibody or antigen-binding portion thereof that specifically binds a phosphorylated NF45 protein. The present invention also relates to methods of utilizing these antibodies to determine the therapeutic efficacy of a candidate compound and methods for screening for candidate compounds that increase the phosphorylation of NF45 protein in a cell.

### CROSS-REFERENCE TO RELATED APPLICATIONS

This application claims priority to, and the benefit of, U.S. Provisional Application No. 61/324,964, filed Apr. 16, 2010. This application is incorporated herein by reference in its entirety.

### BACKGROUND OF THE INVENTION

Cancer is the second leading cause of death in the United States, exceeded only by heart disease. (Cancer Facts and Figures 2004, American Cancer Society, Inc.). Despite recent advances in cancer diagnosis and treatment, surgery and radiotherapy may be curative if a cancer is found early, but current drug therapies for metastatic disease are mostly palliative and seldom offer a long-term cure. Even with new chemotherapies entering the market, the need continues for new drugs effective in monotherapy or in combination with existing agents as first line therapy, and as second and third line therapies in treatment of resistant tumors.

Cancer cells are by definition heterogeneous. For example, within a single tissue or cell type, multiple mutational “mechanisms” may lead to the development of cancer. As such, heterogeneity frequently exists between cancer cells taken from tumors of the same tissue and same type that have originated in different individuals. Frequently observed mutational “mechanisms” associated with some cancers may differ between one tissue type and another (e.g., frequently observed mutational “mechanisms” leading to colon cancer may differ from frequently observed “mechanisms” leading to leukemias). It is therefore often difficult to predict whether a particular cancer will respond to a particular chemotherapeutic agent (Cancer Medicine, 5^{th} edition, Bast et al., B. C. Decker Inc., Hamilton, Ontario).

Components of cellular signal transduction pathways that regulate the growth and differentiation of normal cells can, when dysregulated, lead to the development of cellular proliferative disorders and cancer. Mutations in cellular signaling proteins may cause such proteins to become expressed or activated at inappropriate levels or at inappropriate times during the cell cycle, which in turn may lead to uncontrolled cellular growth or changes in cell-cell attachment properties. For example, dysregulation of receptor tyrosine kinases by mutation, gene rearrangement, gene amplification, and overexpression of both receptor and ligand has been implicated in the development and progression of human cancers.

Accordingly, there is a need in the art to identify cellular signaling proteins which may be dysregulated during the development and progression of cellular proliferation disorders and cancer, and to utilize these proteins as biomarkers for disease progression and therapeutic treatment efficacy. The present invention addresses these needs.

### SUMMARY OF THE INVENTION

The present invention provides, in part, an isolated antibody or antigen-binding portion thereof that specifically binds a protein comprising the amino acid sequence of SEQ ID NO: 1 phosphorylated at one or more serine, threonine, or tyrosine residues. Preferably, the isolated antibody specifically binds a protein of SEQ ID NO: 1 when phosphorylated at a threonine residue. More preferably, the isolated antibody specifically binds a protein of SEQ ID NO: 1 when phosphorylated at threonine residue T 388. Most preferably, the isolated antibody only binds the protein when phosphorylated at threonine residue T 388 and not phosphorylated at any other amino acid residues.

The present invention also provides, in part, an isolated peptide comprising the amino acid sequence of SEQ ID NO: 1 phosphorylated at one or more serine, threonine, or tyrosine residues. Preferably, the isolated peptide is phosphorylated at one or more threonine residues. More preferably, the isolated peptide is phosphorylated at threonine residue T 388 of SEQ ID NO: 1. Most preferably, the isolated peptide is phosphorylated only at threonine residue T 388 and not phosphorylated at any other amino acid residues. The present invention also provides an isolated peptide comprising, consisting essential of, or consisting of, the amino acid sequence of SED ID NO: 2 phosphorylated at threonine residue T 6.

The present invention also provides, in part, a method including (a) contacting a biological sample with an isolated antibody provided by the instant invention and described herein; and, (b) assessing a complex formed between a peptide comprising the amino acid sequence of SEQ ID NO:1 phosphorylated at one or more serine, threonine, or tyrosine residues and said antibody to determine the presence, absence, or amount of said peptide in said biological sample.

The present invention also provides, in part, a method comprising (a) exposing a subject to a candidate compound; (b) obtaining a biological sample from said subject following said exposure; (c) determining the presence, absence, or amount of a peptide comprising the amino acid sequence of SEQ ID NO:1 phosphorylated at one or more serine, threonine, or tyrosine residues in said biological sample; and, (d) comparing the presence, absence, or amount of said peptide in said biological sample with the presence, absence, or amount of said peptide determined in a biological sample obtained from a subject not exposed to the candidate compound.

The present invention also provides, in part, a method comprising (a) contacting a biological sample with a candidate compound; (b) determining the presence, absence, or amount of a peptide comprising the amino acid sequence of SEQ ID NO:1 phosphorylated at one or more serine, threonine, or tyrosine residues in said biological sample following contact with the candidate compound; and, (c) comparing the presence, absence, or amount of said peptide in said biological sample with the presence, absence, or amount of said peptide determined in a biological sample not contacted with the candidate compound.

Determining the presence, absence, or amount of a peptide comprising the amino acid sequence of SEQ ID NO:1 phosphorylated at one or more serine, threonine, or tyrosine residues in said biological sample can include contacting said biological sample with an isolated antibody or antigen-binding portion thereof that specifically binds to said peptide comprising the amino acid sequence of SEQ ID NO:1 phosphorylated at one or more serine, threonine, or tyrosine residues and assessing a complex formed between said peptide and said antibody. The antibody can specifically bind to the peptide phosphorylated at one or more threonine residues. More preferably, the antibody specifically binds to the peptide phosphorylated at threonine residue T388. The detection step can include flow cytometry, an immunohistochemical assay, ELISA or a Western Blot. The sample can be any biological sample. Preferably the sample is a cell or tissue sample. The subject can be a mammal. Preferably, the subject is a human.

The methods of the present invention further include, in part, identifying a candidate compound. The candidate compound can be a compound capable of inducing or increasing the presence of a peptide comprising the amino acid sequence of SEQ ID NO:1 phosphorylated at one or more serine, threonine, or tyrosine residues in the subject or sample. Inducing or increasing the presence of a peptide comprising the amino acid sequence of SEQ ID NO:1 phosphorylated at one or more serine, threonine, or tyrosine residues includes inducing or increasing the phosphorylation state or level of a peptide comprising the amino acid sequence of SEQ ID NO: 1, when compared to the absence of the candidate compound. The candidate compound of the present invention can be a topoisomerase inhibitor. More preferably, a topoisomerase I inhibitor. The candidate compound of the present invention can be a quinone, naptho-quinone or ortho-quinone. The candidate compound is a lapachone or an analog or derivative thereof or a pharmaceutically acceptable salt, prodrug, metabolite, polymorph or solvate thereof. Preferably, the candidate compound is β-Lapachone or an analog or derivative thereof or (S)-1′-(3-(4-(tert-butyl)phenoxy)-2-hydroxypropyl)-2H-spiro[naphtho[1,2-b][1,4]oxathiine-3,4′-piperidine]-5,6-dione or an analog or derivative thereof. More preferably, the candidate compound is a prodrug of β-Lapachone. Most preferably, the candidate compound is a

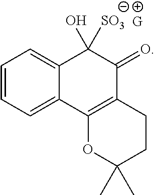

The methods of the present invention further include, in part, identifying a subject having an induction or increase in a peptide comprising the amino acid sequence of SEQ ID NO:1 phosphorylated at one or more serine, threonine, or tyrosine residues following exposure to a candidate compound and administering a chemotherapeutic agent or radiation therapy to said subject. The subject can be a mammal. Preferably, the subject is a human. The subject can be suffering from, or diagnosed with, a cell proliferation disorder. The cell proliferation disorder can be cancer or pre-cancer. Preferably, the administration of the candidate compound, the chemotherapeutic agent or the radiation therapy, alone or in any combination thereof, treats the proliferation disorder in said subject

The present invention also provides, in part, a method for inducing or increasing the amount of a peptide comprising the amino acid sequence of SEQ ID NO:1 phosphorylated at one or more serine, threonine, or tyrosine residues in a hyper-proliferating cell comprising contacting the cell with candidate compound. Preferably, the isolated peptide is phosphorylated at one or more threonine residues. More preferably, the isolated peptide is phosphorylated at threonine residue T 388 of SEQ ID NO: 1. Most preferably, the isolated peptide is phosphorylated only at threonine residue T 388 and not phosphorylated at any other amino acid residues. The cell can be in vivo, in vitro or ex vivo. The cell can be a cancer cell or pre-cancer cell.

The present invention also provides, in part, methods for producing an antibody of the invention. One method includes: (a) introducing an isolated peptide of the invention to a mammal in an amount sufficient to produce an antibody to said peptide; and (b) recovering said antibody from said mammal. Another method includes: (a) introducing an isolated peptide of the invention to a mammal in an amount sufficient to elicit lymphocytes that are capable of producing an antibody to said peptide; (b) recovering said lymphocytes from said mammal; (c) fusing said lymphocytes with myeloma cells; (d) culturing said hybridoma cells under appropriate growth conditions; and (e) recovering one or more monoclonal antibodies to said peptide from the cell culture media The method can further comprise purifying the one or more monoclonal antibodies recovered in step (e). Preferably the isolated peptide comprises the amino acid sequence of SEQ ID NO: 1 phosphorylated at one or more serine, threonine, or tyrosine residues. More preferably, the isolated peptide is phosphorylated at one or more threonine residues. Most preferably, the isolated peptide is phosphorylated at threonine residue T 388 of SEQ ID NO: 1 or an isolated peptide consisting of the amino acid sequence of SED ID NO: 2 phosphorylated at threonine residue T 6.

Unless otherwise defined, all technical and scientific terms used herein have the same meaning as commonly understood by one of ordinary skill in the art to which this invention belongs. In the specification, the singular forms also include the plural unless the context clearly dictates otherwise. Although methods and materials similar or equivalent to those described herein can be used in the practice or testing of the present invention, suitable methods and materials are described below. All publications, patent applications, patents, and other references mentioned herein are incorporated by reference. The references cited herein are not admitted to be prior art to the claimed invention. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a photograph of an immunoblot showing a phosphorylated protein with a MW of approximately 45 kD.

FIG. 2, Panel A is a photograph of an immunoblot showing increase NF45 phosphorylation in the presence of β-Lapachone. Panel B is a photograph of an immunoprecipitation assay using anti-NF90 and anti-pNF45 antibodies. Panel C is a photograph of an immunoblot showing that knockdown of NF45 abolished phosphorylation of NF45.

FIG. 3, Panel A is a photograph of an immunoblot showing the induction of pNF45 (T388) in a time- and concentration-dependent manner following treatment with 3-Lapachone at various times. Panel B is a photograph of an immunoblot showing the induction of pNF45 (T388) in a time- and concentration-dependent manner following treatment with β-Lapachone at various concentrations.

FIG. 4, Panel A is a series of photographs showing cellular staining of phosphorylated NF45 following treatment with β-Lapachone at various times in MIA PaCa-2 cells. Panel B is a series of photographs showing cellular staining of non-phosphorylated NF45 following treatment with β-Lapachone at various times in MIA PaCa-2 cells.

FIG. 5 is a photograph of a series of immunoblots showing the phosphorylation of NF45 following treatment with or without doxorubicin, cisplatin or camptothecin for various times in MIA PaCa-2 cells.

FIG. 6 is a photograph of a series of immunoblots showing the effect of ATM and DNA-PK inhibitors on NF45 phosphorylation by β-Lapachone in MIA PaCa-2 cells.

### DETAILED DESCRIPTION OF THE INVENTION

Human nuclear factor 45 kD (NF45), also known as Interleukin enhancer-binding factor 2 (ILF2), along with human nuclear factor 90 (NF90), also known as Interleukin enhancer-binding factor 3 (ILF3), were originally purified as a sequence-specific DNA binding complex regulating the interleukin-2 (IL-2) promoter. NF45 associates with NF90 in the nucleus and regulates IL-2 gene transcription at the antigen receptor response element (ARRE)/NF-AT DNA target sequence (Kao et al., J. Biol. Chem. 269 (1994) 20691-20699). NF45 is widely expressed in normal tissues, especially testis, brain, and kidney, with a predominantly nuclear distribution. NF45 mRNA expression is increased in lymphoma and leukemia cell lines. The human and murine NF45 proteins are 390 amino acid residues in length and differ only by substitution of valine by isoleucine at amino acid residue 142. The amino acid sequence for Human NF45 is shown in Table 1.

TABLE 1
Amino Acid Sequence of Human NF45 (SEQ ID NO: 1)

| | |
|---|---|
| 1 | mrgdrgrgrg grfgsrggpg ggfrpfvphi pfdfylcema fprvkpapde tsfseallkr |
| 61 | nqdlapnsae qasilslvtk innvidnliv apgtfevqie evrqvgsykk gtmttghnva |
| 121 | dlvvilkilp tleavaalgn kvveslraqd psevltmltn etgfeisssd atvkilittv |
| 181 | ppnlrkldpe lhldikvlqs alaairharw feenasqstv kvlirllkdl rirfpgfepl |
| 241 | tpwildllgh yavmnnptrq plalnvayrr clqilaaglf lpgsvgitdp cesgnfrvht |
| 301 | vmtleqqdmv cytaqtlvri lshggfrkil gqegdasyla seistwdgvi vtpsekayek |
| 361 | ppekkegeee eenteeppqg eeeesmetqe |

Peptides identified by mass spectrometry are underlined in Table 1. Potential ATM or ATR phosphorylation sites are shown in italics in Table 1.

NF45 mRNA and protein sequences in GenBank for various species are listed in Table 2.

TABLE 2

| | mRNA sequence | Protein sequence |
|---|---|---|
| Human | NM_004515.2 | NP_004506.2 |
| Chimpanzee | XM_001142693.1 | XP_001142693.1 |
| | XM_001142161.1 | XP_001142161.1 |
| | XM_513823.2 | XP_513823.2 |
| | XM_001142492.1 | XP_001142492.1 |
| | XM_001142785.1 | XP_001142785.1 |
| | XM_001142410.1 | XP_001142410.1 |
| Orangutan | NM_001132083.1 | NP_001125555.1 |
| Monkey | XM_001111422.2 | XP_001111422.1 |
| Common Marmoset | XM_002759988.1 | XP_002760034.1 |
| Mouse | NM_026374.3 | NP_080650.1 |
| Rat | NM_001047886.1 | NP_001041351.1 |
| Cow | NM_001038187.1 | NP_001033276.1 |
| Dog | XM_537263.2 | XP_537263.1 |
| | XM_860930.1 | XP_866023.1 |
| Horse | XM_001495039.2 | XP_001495089.1 |
| Chicken | XM_423437.2 | XP_423437.2 |
| Frog | NM_203666.1 | NP_988997.1 |
| | NM_001087106.1 | NP_001080575.1 |
| Zebrafish | NM_213236.1 | NP_998401.1 |

Fluorescence in situ hybridization localized the human NF45 gene to chromosome 1q21.3, and mouse NF45 gene to chromosome 3F1. Promoter analysis of 2.5 kB of the murine NF45 gene reveals that significant activation is conferred by factors, possibly including NF-Y, that bind to the CCAAT-box sequence. The function of human NF45 in regulating IL-2 gene expression was characterized in Jurkat T-cells stably transfected with plasmids directing expression of NF45 cDNA in sense or antisense orientations. NF45 sense expression increased IL-2 luciferase reporter gene activity 120-fold, and IL-2 protein expression 2-fold compared to control cells. NF45 is a highly conserved, regulated transcriptional activator, and one target gene is IL-2 (Zhao et al., Exp Cell Res. 305(2):312-23, 2005).

Transfection assays showed that NF45 binds NF90 strongly and stimulates its ability to activate but not to inhibit gene expression (Reichman et al., Mol. Cell. Biol. 22(1):343-56, 2002). NF90 forms heterodimeric core complexes with NF45 and large in vivo complexes. Depletion of NF45 led to decreased levels of NF90. This coregulation is at the protein level: the stability of NF90 family proteins is dependent on binding to their NF45 partner. Cell growth is retarded and giant multinucleated cells accumulate when the expression of NF45 or NF90 is reduced in HeLa cells.

Various compounds are well known in the art for their anti-proliferative and anti-cancer activities. One group of compounds known as quinones (including ortho-quinones and ortho-napthoquinones) are effective anti-proliferative agents. The quinones are a large and varied group of natural products found in all major groups of organisms. Quinones are a group of aromatic dioxo compounds derived from benzene or multiple-ring hydrocarbons such as naphthalene, anthracene, etc. They are classified as benzoquinones, naphthoquinones, anthraquinones, etc., on the basis of the ring system. The C═O groups are generally ortho or para, and form a conjugated system with at least two C═C double bonds; hence the compounds are colored, yellow, orange or red. Quinones with long isoprenoid side chains, such as plastoquinone, ubiquinone and phytoquinone are involved in the basic life processes of photosynthesis and respiration. Quinones are biosynthesized from acetate/malonate via shikimic acid.

Many efficient antineoplastic drugs are either quinones (anthracycline derivatives, mitoxantrone, actinomycin), quinonoid derivatives (quinolones, genistein, bactracyclin), or drugs such as etoposide that can easily be converted to quinones by in vivo oxidation. Gantchev et al. (1997) Biochem. Biophys. Res. Comm 237:24-27. The literature on quinone-DNA interactions is replete with references to quinones having the potential to undergo redox cycling with the formation of highly reactive oxygen species that are thought to relate to their cytotoxicity. O'Brien (1991) Chem. Biol. Interactions 80:1-41. It has also been shown that many quinones are efficient modifiers of the enzymatic activity of topoisomerase II, an enzyme essential for cell division.

Quinones are now widely used as anti-cancer, anti-bacterial and anti-malarial drugs, as well as fungicides. The antitumor activities of the quinones were revealed more than two decades ago when the National Cancer Institute published a report in which fifteen-hundred synthetic and natural quinones were screened for their anticancer activities. Driscoll et al. (1974) Cancer Chemot. Reports 4:1-362. Anti-cancer quinones include β-Lapachone, a plant product, which inhibits DNA topoisomerase II and induces cell death with characteristics of apoptosis in human prostate and promyelocytic leukemia cancer cell lines. Human breast and ovary carcinoma showed sensitivity of the cytotoxic effect of β-Lapachone without signs of apoptosis. Li et al. (1995) Cancer Res. 55:3712-5; and Planchon et al. (1995) Cancer Res. 55:3706-11. 1,2-Naphthoquinone (3,4-b)dihydrofuran inhibits neoplastic cell growth and proliferation of several cancers, such as prostate, breast, colon, brain and lung, including multi-drug resistant types. WO 97/31936. Furano-naphthoquinone derivatives and other naphthoquinones and naphth-[2,3-d]-imidazole-4,9-dione compounds are also useful in treating malignant tumors such as those affecting the blood, breast, central nervous system, cervix, colon, kidney, lung, prostate and skin. WO 97/30022 and JP Patent No. 9235280. Anthraquinone derivatives with telomerase inhibitory activity are also useful in treating leukemia, lung cancer, myeloma, lymphoma, prostate, colon, head and neck, melanoma, hepatocellular carcinoma, bladder, ovarian, breast and gastric cancers. WO 98/25884 and WO 98/25885. Ansamycin benzoquinones are useful in the treatment of primitive neuroectodermal tumors, prostate cancer, melanoma and metastatic Ewing's sarcoma. WO 94/08578.

Quinones also have a number of other medicinal uses. Terpenoid-type quinones are also useful as treatments for diabetes. U.S. Pat. No. 5,674,900. Additional quinones can be used to treat cirrhosis and other liver disorders. U.S. Pat. Nos. 5,210,239 and 5,385,942. Hydroquinone amines and quinone amines are also useful for treating a number of conditions, including spinal trauma and head injury. U.S. Pat. No. 5,120,843. Degenerative central nervous system diseases, as well as vascular diseases, are treatable with quinones such as Idebenone[2,3-dimethoxy-5-methyl-6-(10-hydroxydecyl)-1,4-benzoquinone] and Rifamycin S. Mordente et al. (1998) Chem. Res. Toxicol. 11:54-63; Rao et al. (1997) Free Radic. Biol. Med. 22:439-46; Cortelli et al. (1997) J. Neurol. Sci. 148:25-31; and Mahadik et al. (1996) Prostaglandins Leukot. Essent. Fatty Acids 55:45-54. A vitamin K analog, 6-cyclo-octylamino-5,8-quinoline quinone shows efficacy for treatment of leprosy and tuberculosis. U.S. Pat. No. 4,963,565. Hydroquinone is used to treat skin pigmentation disorders. Clarys et al. (1998) J. Dermatol. 25:412-4. Mitomycin C-related drug indoloquinone EO9 has demonstrated cell killing against HL-60 human leukemia cells, H661 human lung cancer cells, rat Walker tumor cells and human HT29 colon carcinoma cells. Begleiter et al. (1997) Oncol. Res. 9:371-82; and Bailey et al. (1997) Br. J. Cancer 76:1596-603. Quinones such as aloin, a C-glycoside derivative of anthraquinone, accelerate ethanol oxidation and may be useful in treating acute alcohol intoxication. Chung et al. (1996) Biochem. Pharmacol. 52:1461-8 and Nanji et al. (1996) Toxicol. Appl. Pharmacol. 140:101-7. Quinones capsaicin and resiniferatoxin blocked activation of nuclear transcription factor NF-κB, which is required for viral replication, immune regulation and induction of various inflammatory and growth-regulatory genes. Singh et al. (1996) J. Immunol. 157:4412-20. Antiretroviral and antiprotozoan naphthoquinones are described in U.S. Pat. Nos. 5,780,514 and 5,783,598. Anthraquinones are also useful as laxatives. Ashraf et al. (1994) Aliment. Pharmacol. Ther. 8:329-36; and Muller-Lissner (1993) Pharmacol. 47 (Suppl. 1): 138-45.

Quinones can be administered alone or in conjunction with other agents, such as 1,2-dithiole-3-thione. Begleiter et al. (1997). Hydroxyquinone can be used in conjunction with glycol or glyceryl esters of retinoic acid to treat skin disorders. WO 9702030. Combinational chemotherapy of carboquone, a benzoquinine derivative, and cis-Platinum, diminishes the side effects of the former. Saito (1988) Gan To Kagaku Ryoho 15:549-54.

Quinones also have various additional uses. A few quinones are used as laxatives and worming agents, and others are used as pigments in cosmetics, histology and aquarrell paints. Quinones include 2,5-cyclohexadiene-1,4-dione, which is useful as an oxidizing agent; in photography (U.S. Pat. No. 5,080,998); in manufacturing dyes and hydroquinone; in tanning hides; in strengthening animal fibers; and as a reagent.

In rapidly dividing cells such as tumor cells, cytotoxicity due to quinone administration has been attributed to DNA modification. However the molecular basis for the initiation of quinone cytotoxicity in resting or non-dividing cells has been attributed to the alkylation of essential protein thiol or amine groups and/or the oxidation of essential protein thiols by activated oxygen species and/or GSSG, glutathione disulfide. Oxidative stress arises when the quinone is reduced by reductases to a semiquinone radical which reduces oxygen to superoxide radicals and reforms the quinone. This futile redox cycling and oxygen activation forms cytotoxic levels of hydrogen peroxide and GSSG is retained by the cell and causes cytotoxic mixed protein disulfide formation. O'Brien (1991) Chem. Biol. Interact. 80:1-41.

Many quinone compounds and methods for use of these quinone compounds in treating diseases are well described in U.S. Pat. No. 6,482,943.

Additionally, a subset of quinones (e.g., various ortho-quinone and ortho-naphthoquinone derivatives), with anti-proliferative activity are well described in U.S. Pat. Nos. 5,969,163 and 5,824,700.

The most promising subset of quinines with anti-proliferative activity is the subset of quinones designated lapachones which are well described in U.S. Pat. No. 5,763,625.

β-Lapachone (3,4-dihydro-2,2-dimethyl-2H-naphtho[1,2-b]pyran-5,6-dione) is a simple non-water soluble orthonapthoquinone, which was first isolated in 1882 by Paterno from the heartwood of the lapacho tree (See Hooker, S C, (1936)I. Am. Chem. Soc. 58:1181-1190; Goncalves de Lima, 0, et al., (1962) Rev. Inst. Antibiot. Univ. Recife. 4:3-17). The structure of β-Lapachone was established by Hooker in 1896 and it was first synthesized by Fieser in 1927 (Hooker, S C, (1936) I. Am. Chem. Soc. 58:1181-1190). Lapachol and β-lapachone (with numbering) have the following chemical structures:

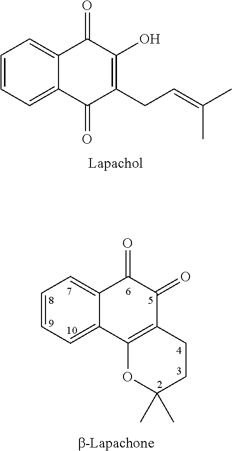

β-Lapachone can, for example, be obtained by simple sulfuric acid treatment of the naturally occurring lapachol, which is readily isolated from Tabebuia avellenedae growing mainly in Brazil, or is easily synthesized from seeds of lomatia growing in Australia (Li, C J, et al., (1993) J. Biol. Chem. 268:22463-33464). Methods for formulating β-Lapachone can be accomplished as described in U.S. Pat. Nos. 6,962,944 and 7,074,824. Methods for synthesizing β-Lapachone can be accomplished as described in U.S. Pat. No. 6,458,974.

As used herein, derivatives or analogs of β-Lapachone include, for example, 3,4-dihydro-2,2-dimethyl-3-(3-methyl-2-butenyl)-2H-naphtho[1,2-b]pyran-5,6-dione, 3,4-dihydro-2,2-dimethyl-2H-naphtho[1,2-b]thiopyran-5,6-dione and 3,4-dihydro-4,4-dimethyl-2H-naphtho[1,2-b]thiopyran-5,6-dione.

Other derivatives or analogs of β-lapachone are described in PCT International Application PCT/US93/07878 (WO94/04145), and U.S. Pat. No. 6,245,807. PCT International Application PCT/US00/10169 (WO 00/61142), discloses β-lapachone, which may have a variety of substituents at the 3-position as well as in place of the methyl groups attached at the 2-position. U.S. Pat. Nos. 5,763,625, 5,824,700, and 5,969,163, disclose analogs and derivatives with a variety of substituents at the 2-, 3- and 4-positions. Furthermore, a number of journals report β-lapachone analogs and derivatives with substituents at one or more of the following positions: 2-, 3-, 8- and/or 9-positions, (See, Sabba et al., (1984) J Med Chem 27:990-994 (substituents at the 2-, 8- and 9-positions); (Portela and Stoppani, (1996) Biochem Pharm 51:275-283 (substituents at the 2- and 9-positions); Goncalves et al., (1998) Molecular and Biochemical Parasitology 1:167-176 (substituents at the 2- and 3-positions)).

U.S. Patent Application Publication No. 2004/0266857 and PCT International Application PCT/US2003/037219 (WO 04/045557), incorporated by reference herein, disclose and several journal reports describe structures having sulfur-containing hetero-rings in the “α” and “β” positions of lapachone (Kurokawa S, (1970) Bulletin of The Chemical Society of Japan 43:1454-1459; Tapia, R A et al., (2000) Heterocycles 53(3):585-598; Tapia, R A et al., (1997) Tetrahedron Letters 38(1):153-154; Chuang, C P et al., (1996) Heterocycles 40(10):2215-2221; Suginome H et al., (1993) Journal of the Chemical Society, Chemical Communications 9:807-809; Tonholo J et al., (1988) Journal of the Brazilian Chemical Society 9(2):163-169; and Krapcho A P et al., (1990) Journal of Medicinal Chemistry 33(9):2651-2655).

β-lapachone is an agent with a reported anti-cancer activity in a limited number of cancers. For example, there is reported a method and composition for the treatment of tumors, which comprises the administration of an effective amount of β-lapachone, in combination with a taxane derivative (U.S. Pat. No. 6,664,288; WO00/61142). Additionally, U.S. Pat. No. 6,245,807 discloses the use of β-lapachone, amongst other β-lapachone derivatives, for use in treatment of human prostate disease. As a single agent, β-Lapachone has also been reported to decrease the number of tumors, reduce tumor size, or increase the survival time, or a combination of these, in xenotransplant mouse models of human ovarian cancer (Li, C. J. et al., (1999) Proc. Natl. Acad. Sci. USA, 96(23): 13369-13374), human prostate cancer (Li, C. J. et al., (1999) Proc. Natl. Acad. Sci. USA, 96(23): 13369-13374), human breast cancer (Li, C. J. et al., (2000) AACR Proc., p. 9), and human multiple myeloma (U.S. Pat. No. 7,070,797; WO 03/011224).

β-lapachone appears to work by activating DNA damage response/checkpoint pathways, which may involve unscheduled expression of checkpoint molecules, e.g. E2F1, independent of DNA damage and cell cycle stages. Several studies have shown that β-lapachone activates checkpoint pathways and induces cell death in cancer cells from a variety of tissues without causing death of normal cells from these tissues (U.S. Patent Application Publication No. 2002/0169135, incorporated by reference herein). In normal cells with their intact regulatory mechanisms, such an imposed expression of a checkpoint molecule results in a transient expression pattern and causes little consequence. In contrast, cancer and pre-cancer cells have defective mechanisms. Drug-induced elevation of checkpoint molecules, e.g. E2F1, can lead to selective cell death in these dysregulated cells.

Moreover, PCT Application PCT/US06/20780, incorporated by reference herein, discloses tricyclic spiro-oxathiine naphthoquinone derivatives, a synthetic method for making the derivatives, and the use of the derivatives to induce cell death and/or to inhibit proliferation of cancer or precancerous cells. The naphthoquinone derivatives of the present invention are related to β-lapachone. WO 2006/128120, incorporated by reference herein, discloses sulfur analogs and derivatives of β-lapachone as well as methods of use thereof. These compounds can be used in pharmaceutical compositions for the treatment or prevention of cell proliferation disorders.

In addition to their antineoplastic uses, quinones also have a number of other medicinal uses. Terpenoid-type quinones are also useful as treatments for diabetes. U.S. Pat. No. 5,674,900. Additional quinones can be used to treat cirrhosis and other liver disorders. U.S. Pat. Nos. 5,210,239 and 5,385,942.

Hydroquinone amines and quinone amines are also useful for treating a number of conditions, including spinal trauma and head injury. U.S. Pat. No. 5,120,843. Degenerative central nervous system diseases, as well as vascular diseases, are treatable with quinones such as Idebenone[2,3-dimethoxy-5-methyl-6-(10-hydroxydecyl)-1,4-benzoquinone] and Rifamycin (S. Mordente et al. (1998) Chem. Res. Toxicol. 11:54-63; Rao et al. (1997) Free Radic. Biol. Med. 22:439-46; Cortelli et al. (1997) J. Neurol. Sci. 148:25-31; and Mahadik et al. (1996) Prostaglandins Leukot. Essent. Fatty Acids 55:45-54). A vitamin K analog, 6-cyclo-octylamino-5,8-quinoline quinone, shows efficacy for treatment of leprosy and tuberculosis. (U.S. Pat. No. 4,963,565). Hydroquinone is also used to treat skin pigmentation disorders. Clarys et al. (1998) J. Dermatol. 25:412-4. Mitomycin C-related drug indoloquinone EO9 has demonstrated cell killing against HL-60 human leukemia cells, H661 human lung cancer cells, rat Walker tumor cells and human HT29 colon carcinoma cells (Begleiter et al. (1997) Oncol. Res. 9:371-82; and Bailey et al. (1997) Br. J. Cancer 76:1596-603).

Quinones such as aloin, a C-glycoside derivative of anthraquinone, accelerate ethanol oxidation and may be useful in treating acute alcohol intoxication. (Chung et al. (1996) Biochem. Pharmacol. 52:1461-8 and Nanji et al. (1996) Toxicol. Appl. Pharmacol. 140:101-7). Quinones capsaicin and resiniferatoxin blocked activation of nuclear transcription factor NF-κB, which is required for viral replication, immune regulation and induction of various inflammatory and growth-regulatory genes (Singh et al. (1996) J. Immunol. 157:4412-20). Antiretroviral and antiprotozoan naphthoquinones are described in U.S. Pat. Nos. 5,780,514 and 5,783,598. Anthraquinones are also useful as laxatives (Ashraf et al. (1994) Aliment. Pharmacol. Ther. 8:329-36; and Muller-Lissner (1993) Pharmacol. 47 (Suppl. 1): 138-45).

Because of the wide variety of biological processes in which quinones play a critical role, it would be advantageous to develop novel quinones for various uses, including disease treatment. One obstacle, however, to the development of pharmaceutical formulations comprising quinones, such as β-lapachone or β-lapachone analogs, for pharmaceutical use is the low solubility of many quinone compounds, including β-lapachone compounds, in pharmaceutically acceptable solvents. There are also drawbacks related to the pharmacokinetic profiles of traditional formulations comprising quinones.

U.S. Pat. Nos. 6,962,944 and 7,074,824 disclose pharmaceutical compositions comprising a therapeutically effective amount of β-lapachone, or a derivative or analog thereof, and a pharmaceutically acceptable solubilizing carrier molecule, which may be a water-solubilizing carrier molecule such as hydroxypropyl-β-cyclodextrin, or an oil-based solubilizing carrier molecule, for enhancing the solubility of β-lapachone in aqueous solution. The therapeutically effective amount of β-lapachone, or a derivative or analog thereof, may be complexed with the pharmaceutically acceptable solubilizing carrier molecule in aqueous solution.

Additional U.S. patents and U.S. patent Publications describe various quinone and lapachone derivatives.

U.S. Pat. No. 7,902,354 discloses compounds of the following formula:

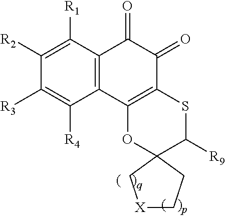

or a pharmaceutically acceptable salt and/or an individual diastereomer thereof,

wherein:

X═N-J₁,

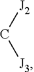

### O, or S;

p=0, 1 or 2;

q=p or p+1, provided that if p is 0, q does not equal p;

R₁, R₂, R₃, and R₄ are each, independently, H, OH, F, Cl, Br, I, CH₃, CF₃, C₂-C₆ straight chain alkyl, substituted C₁-C₆ straight chain alkyl, C₃-C₆ branched alkyl, C₃-C₈ cycloalkyl, allyl, C₂-C₆ straight chain alkenyl, substituted C₂-C₆ straight chain alkenyl, C₃-C₆ branched alkenyl, C₅-C₈ cycloalkenyl, C₂-C₆ alkynyl, NO₂, CN, NH₂, alkylamine, substituted alkylamine, dialkylamine, arylamine, C(O)NHR₁₄, NHC(O)R₁₅, carbamoyl, aminesulfoxide, sulfonamide, sulfamoyl, sulfonic acid, phenyl, C₅-C₈ aryl, heteroaryl, heterocyclyl, OCH₃, OCF₃, C₂-C₆ alkoxy, alkoxycarbonyl, carboxyacid, carbonylalkoxy, SH, thioalkyl, thioaryl, alkylthioaryl, or C₁-C₆ hydroxyl alkyl;

J₁ is —(CR₅R₆)ₙ—(CR₇R₈)ₘ—Y, —S(O)ₒ—Z, amidine, substituted amidine, heterocyclyl, substituted heterocyclyl, 3,4-dioxo-3,4-dihydronaphthalenyl, heteroaryl, substituted heteroaryl,

or

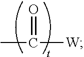

m=0, 1, 2, 3, 4, or 5;

n=0, 1, 2, 3, 4, or 5;

o=1 or 2;

t=1 or 2;

R₅ and R₆ are each, independently, H, OH, CH₃, CF₃, C₂-C₆ straight chain alkyl, C₃-C₆ branched alkyl, C₃-C₈ cycloalkyl, allyl, C₂-C₆ straight chain alkenyl, C₃-C₆ branched alkenyl, C₅-C₈ cycloalkenyl, C₂-C₆ alkynyl, phenyl, C₅-C₈ aryl, heteroaryl, heterocyclyl, carboxylate, or carbonylalkoxy; when R₅═R₆, R₅ cannot be OH, NH₂, SH, or NO₂;

R₇ and R₈ are each, independently, H, F, Cl, Br, I, OH, CH₃, C₂-C₆ straight chain alkyl, CF₃, C₃-C₆ branched alkyl, C₃-C₈ cycloalkyl, C₂-C₆ alkoxy, allyl, C₂-C₆ straight chain alkenyl, C₃-C₆ branched alkenyl, C₅-C₈ cycloalkenyl, C₂-C₆ alkynyl, NO₂, CN, amine, alkylamine, dialkylamine, arylamine, carbamoyl, aminesulfoxide, sulfonamide, sulfonic acid, phenyl, C₅-C₈ aryl, heteroaryl, heterocyclyl, OCH₃, OCF₃, alkoxycarbonyl, carboxyacid, carbonylalkoxy, SH, thioalkyl, thioaryl, or alkylthioaryl; when R₇═R₈, R₇ is not OH, NH₂, SH, or NO₂;

Y is H, F, Cl, Br, I, CR₁₀═CHR₁₁, CF₃, CH₃, C₂-C₆ straight chain alkyl, substituted C₂-C₆ straight chain alkyl, C₃-C₆ branched alkyl, CH₂OR₁₆, phenyl, substituted phenyl, C₅-C₈ aryl, substituted C₅-C₈ aryl, C₃-C₈ cycloalkyl, substituted C₃-C₈ cycloalkyl, CH₂-heterocycle, C₅-C₈ cycloalkenyl, aryloxy, substituted aryloxy, heteroaryl, substituted heteroaryl, heterocyclyl, substituted heterocyclyl, benzyl, alkylamine, substituted alkylamine, benzylamine, OH, CH₃, CF₃, OCR₁₂═CHR₁₃, C₂-C₆ alkynyl, amine, dialkylamine, arylamine, amide, carbamoyl, aminesulfoxide, sulfamide, sulfamoyl, sulfonic acid, heteroaryloxy, OCH₃, OCF₃, C₂-C₆ alkoxy, alkenoxy, phenoxy, benzyloxy, alkoxycarbonyl, carboxyacid, carboxyalkoxy, carbonylalkyl, thio, alkylthio, thioalkyl, arylthio, thioaryl, alkylthioaryl, or

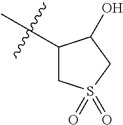

provided that, when n=0 and m=0, Y is H, heterocyclyl, heteroaryl, C₃-C₈ cycloalkyl, C₅-C₈ cycloalkyl, aryl, or

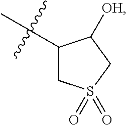

each of which may be substituted; if m=n=o, Y is not

W is C₂-C₆ straight chain alkyl, substituted C₁-C₆ straight chain alkyl, OCH₃, C₂-C₆ alkoxy, alkylthioalkyl, substituted alkylthioalkyl, C₃-C₈ cycloalkyl, substituted C₃-C₈ cycloalkyl, C₅-C₈ aryl, substituted aryl, phenyl, substituted phenyl, CR₁₀═CHR₁₁, alkylthio, benzyl, substituted benzyl, heterocyclyl, substituted heterocyclyl, phenoxy, aryloxy, substituted aryloxy, OCR₁₂═CHR₁₃, benzyloxy, heteroaryloxy, substituted heteroaryloxy, amine, substituted amine, arylamine, substituted arylamine, phenylamine, substituted phenylamine, CH₃, CF₃, C₃-C₆ branched alkyl, C₅-C₈ cycloalkenyl, C₂-C₆ alkynyl, alkylamine, dialkylamine, heteroaryl, CH₂-heterocyclyl, CH₂-substituted heterocyclyl, OCF₃, alkenoxy, CH₂OR₁₆, thioalkyl, arylthio, thioaryl, alkylthioaryl or alkylcarboxy, phenyl sulfonylamide, or substituted aryl sulfonylamide, chlorophenylacetyl;

Z is CH₃, CF₃, C₂-C₆ straight chain alkyl, heteroaryl, substituted heteroaryl, phenyl, substituted phenyl, C₅-C₈ aryl, substituted C₅-C₈ aryl, C₃-C₆ branched alkyl, C₃-C₈ cycloalkyl, C₅-C₈ cycloalkenyl, C₂-C₆ alkynyl, amine, alkylamine, dialkylamine, arylamine, benzyl, heteroaryloxy, heterocyclyl, CH₂-heterocycle, OCH₃, OCF₃, C₂-C₆ alkoxy, alkenoxy, phenoxy, aryloxy or benzyloxy;

R₉ is H, CH₃, C₂-C₆ straight chain alkyl, or C₃-C₆ branched alkyl;

R₁₀, R₁₁, R₁₂, and R₁₃ are each, independently, H, phenyl, C₅-C₈ aryl,

CH₃, CF₃, C₂-C₆ straight chain alkyl, C₃-C₈ cycloalkyl, heteroaryl, or heterocyclyl;

R₁₄ and R₁₅ are each, independently H, C₂-C₆ straight alkyl, C₃-C₆ branched alkyl, C₃-C₈ cycloalkyl, allyl, C₂-C₆ straight alkenyl, branched alkenyl, C₅-C₈ cycloalkenyl, phenyl, C₅-C₈ aryl, benzyl, CH₂C(OCH₃)₂, heteroaryl, or heterocyclyl; and,

R₁₆ is C₃-C₆ branched alkyl, C₅-C₈ aryl, substituted C₅-C₈ aryl, heteroaryl, phenyl, substituted phenyl, CH₂-aryl, benzyl, H, CH₃, CF₃, C₂-C₆ straight chain alkyl, C₃-C₈ cycloalkyl, heterocyclyl, or CH₂-heteroaryl.

J₂ and J₃ are each, independently, H, F, Cl, Br, I, CR₁₇═CHR₁₈, CF₃, CH₃, C₂-C₆ straight chain alkyl, substituted C₁-C₆ straight chain alkyl, C₃-C₆ branched alkyl, CH₂OR₂₁, phenyl, C₅-C₈ aryl, substituted C₅-C₈ aryl, C₃-C₈ cycloalkyl, substituted C₃-C₈ cycloalkyl, CH₂-heterocycle, C₅-C₈ cycloalkenyl, aryloxy, substituted aryloxy, heteroaryl, substituted heteroaryl, heterocyclyl, substituted heterocyclyl, benzyl, alkylamine, substituted alkylamine, benzylamine, OH, CH₃, CF₃, OCR₁₉═CHR₂₀, C₂-C₆ alkynyl, amine, dialkylamine, arylamine, amide, carbamoyl, aminesulfoxide, sulfamide, sulfamoyl, sulfonic acid, heteroaryloxy, OCH₃, OCF₃, C₂-C₆ alkoxy, alkenoxy, phenoxy, benzyloxy, alkoxycarbonyl, carboxyacid, carboxyalkoxy, carbonylalkyl, thio, alkylthio, thioalkyl, arylthio, thioaryl, alkylthioaryl, or

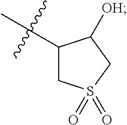

when J₂=J₃, J₂ is not OH, NH₂, SH, or NO₂; J₂ and J₃ can form a 4, 5; 6, 7, 8 membered spiro ring containing 0, 1, or 2 heteroatoms such as O, N, S;

R₁₇, R₁₈, R₁₉, and R₂₀ are each, independently, H, phenyl, C₅-C₈ aryl, CH₃, CF₃, C₂-C₆ straight chain alkyl, C₃-C₈ cycloalkyl, heteroaryl, or heterocyclyl;

R₂₁ is H, C₂-C₆ straight alkyl, C₃-C₆ branched alkyl, C₃-C₈ cycloalkyl, allyl, C₂-C₆ straight alkenyl, branched alkenyl, C₅-C₈ cycloalkenyl, phenyl, C₅-C₈ aryl, benzyl, CH₂C(OCH₃)₂, heteroaryl, or heterocyclyl.

U.S. Pat. No. 7,902,354 discloses the following specific compounds, or a pharmaceutically acceptable salt and/or an individual diastereomer thereof: 1′-(3-chlorobenzoyl)spiro[naphtho[1,2-b][1,4]oxathiine-2,4′-piperidine]-5,6-dione;
1′-(3,4-dichlorobenzoyl)spiro[naphtho[1,2-b][1,4]oxathiine-2,4′-piperidine]-5,6-dione;
4-[(5,6-dioxo-5,6-dihydro-1′H-spiro[naphtho[1,2-b][1,4]oxathiine-2,4′-piperidin]-1′-yl)methyl]benzonitrile;
1′-(2-phenylethyl)spiro[naphtho[1,2-b][1,4]oxathiine-2,4′-piperidine]-5,6-dione;
1′-(4-fluorobenzyl)spiro[naphtho[1,2-b][1,4]oxathiine-2,4′-piperidine]-5,6-dione;
1′-[3-(4-tert-butylphenoxy)-2-hydroxypropyl]spiro[naphtho[1,2-b][1,4]oxathiine-2,4′-piperidine]-5,6-dione;
1′-(2-hydroxy-3-phenylpropyl)spiro[naphtho[1,2-b][1,4]oxathiine-2,4′-piperidine]-5,6-dione;
3-(trifluoromethyl)phenyl 5,6-dioxo-5,6-dihydro-1′H-spiro[naphtho[1,2-b][1,4]oxathiine-2,4′-piperidine-]-1′-carboxylate;
4-fluorophenyl 5,6-dioxo-5,6-dihydro-1′H-spiro[naphtho[1,2-b][1,4]oxathiine-2,4′-piperidine-]-1′-carboxylate;
1′-(2-chloro-6-fluorobenzyl)spiro[naphtho[1,2-b][1,4]oxathiine-2,4′-piperidine]-5,6-dione;
1′-(3-chloro-4-fluorobenzyl)spiro[naphtho[1,2-b][1,4]oxathiine-2,4′-piperidine]-5,6-dione;
1′-(3-phenoxypropyl)spiro[naphtho[1,2-b][1,4]oxathiine-2,4′-piperidine]-5,6-dione;
1′-[2-(4-chlorophenoxy)ethyl]spiro[naphtho[1,2-b][1,4]oxathiine-2,4′-piperidine]-5,6-dione;
1′-[(2S)-3-(4-fluorophenoxy)-2-hydroxypropyl]spiro[naphtho[1,2-b][1,4]oxathiine-2,4′-piperidine]-5,6-dione;
1′-[(2S)-3-(4-tert-butylphenoxy)-2-hydroxypropyl]spiro[naphtho[1,2-b][1,4]oxathiine-2,4′-piperidine]-5,6-dione;
1′-[(2R)-3-(4-tert-butylphenoxy)-2-hydroxypropyl]spiro[naphtho[1,2-b][1,4]oxathiine-2,4′-piperidine]-5,6-dione;
1′-[(2R)-3-(4-fluorophenoxy)-2-hydroxypropyl]spiro[naphtho[1,2-b][1,4]oxathiine-2,4′-piperidine]-5,6-dione;
1′-[(2S)-3-(4-chlorophenoxy)-2-hydroxypropyl]spiro[naphtho[1,2-b][1,4]oxathiine-2,4′-piperidine]-5,6-dione;
1′-[(2R)-3-(4-chlorophenoxy)-2-hydroxypropyl]spiro[naphtho[1,2-b][1,4]oxathiine-2,4′-piperidine]-5,6-dione;
1′-[(2R)-2-hydroxy-3-phenylpropyl]spiro[naphtho[1,2-b][1,4]oxathiine-2,4′-piperidine]-5,6-dione;
1′-[(2S)-2-hydroxy-3-phenylpropyl]spiro[naphtho[1,2-b][1,4]oxathiine-2,4′-piperidine]-5,6-dione;
1′-[(2S)-2-hydroxy-3-(2-methylphenoxy)propyl]spiro[naphtho[1,2-b][1,4]oxathiine-2,4′-piperidine]-5,6-dione;
1′-isopropylspiro[naphtho[1,2-b][1,4]oxathiine-2,4′-piperidine]-5,6-dione;
1′-[(2S)-3-(4-ethylphenoxy)-2-hydroxypropyl]spiro[naphtho[1,2-b][1,4]oxathiine-2,4′-piperidine]-5,6-dione;
1′-[(2R)-3-(4-ethylphenoxy)-2-hydroxypropyl]spiro[naphtho[1,2-b][1,4]oxathiine-2,4′-piperidine]-5,6-dione; and
1′-[(2R)-2-hydroxy-3-(2-methylphenoxy)propyl]spiro[naphtho[1,2-b][1,4]oxathiine-2,4′-piperidine]-5,6-dione.

U.S. Pat. No. 7,902,354 also discloses the compound of formula:

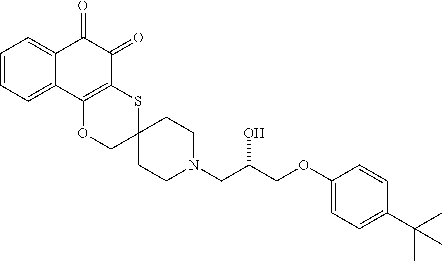

(S)-1′-(3-(4-(tert-butyl)phenoxy)-2-hydroxypropyl)-2H-spiro[naphtho[1,2-b][1,4]oxathiine-3,4′-piperidine]-5,6-dione.

U.S. Pat. No. 7,790,765 discloses compounds of the following formula:

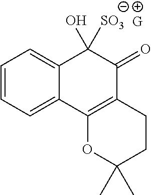

or a pharmaceutically acceptable salt and/or an individual enantiomer/diastereomer thereof; wherein G is a cation. G can be a metal cation. The metal cation can be selected from the group consisting of H⁺, Na⁺, K⁺, Li⁺, and Ca²⁺. The G can also be N⁺(R₁)₄, wherein each R₁ is independently selected from the group consisting of H, C₂-C₆ straight alkyl, C₃-C₆ branched alkyl, C₃-C₈ cycloalkyl, C₅-C₈ cycloalkenyl, phenyl, C₅-C₈ aryl, and benzyl and methods of synthesizing these compounds. U.S. Pat. No. 7,790,765 also discloses the specific compound:

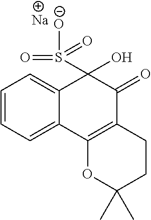

or a pharmaceutically acceptable salt and/or an individual enantiomer/diastereomer thereof.

U.S. Pat. No. 7,812,051 discloses polymer modified lapachone compounds. Specifically, U.S. Pat. No. 7,812,051 discloses compositions comprising a carboxyl-containing polymer associated, via a linking agent of formula (I), with one or more carbonyl-containing or quinone-containing therapeutic agents, wherein said linking agent of formula (I) is associated with said one or more carbonyl-containing or quinone-containing therapeutic agents by an imine bond; wherein said linking agent of formula (I) is of the form

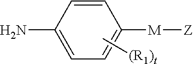

wherein

M is selected from the group consisting of: —(C₁-C₈) alkyl-, —(CH₂)_{q}—O—(CH₂)ᵣ—, —C(═O)—O—(CH₂)ᵣ—, —(C₃-C₇)cycloalkyl-, -aryl-C(═O)—O—(CH₂)ᵣ—, —C(═O)—O-aryl-(CH₂)ᵣ—, -heteroaryl-C(═O)—O—(CH₂)ᵣ—, and —C(═O)—O-heteroaryl-(CH₂)ᵣ—;

Z is —OH, a protected amine, or a protected hydroxyl;

each R₁ is independently selected from the group consisting of: hydrogen, halogen, and —(C₁-C₄) alkyl;

q is from 0-6;

r is from 2-6; and

t is from 0-4. Preferably, the carbonyl-containing or quinone-containing therapeutic agent is β-Lapachone.

U.S. Pat. No. 7,812,051 also discloses compositions comprising a carboxyl-containing polymer associated, via a linking agent of formula (II), with one or more quinone-containing therapeutic agents, wherein said linking agent of formula (II) is associated with said one or more quinone-containing therapeutic agents by a quinol-ester;

wherein said linking agent of formula (II) is of the form

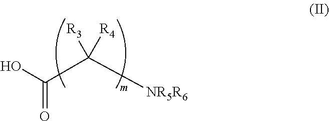

where

each R₃ and R₄ are independently selected from the group consisting of hydrogen, —(C₁-C₈) alkyl, —O—(C₁-C₈) alkyl, —(C₁-C₄) alkyl-aryl, aryl, and heteroaryl;

R₅ is selected from the group consisting of hydrogen, —(C₁-C₈) alkyl, —(C₁-C₈) fluoroalkyl, aryl, and heteroaryl;

R₆ is selected from the group consisting of tert-butoxycarbonyl and CBZ; and

m is from 1 to 8;

alternatively, when m is 1, R₄ and R₅ may be taken together with the carbon and nitrogen atoms bearing them to form a 4 to 7 membered nitrogen-containing heterocycle.

U.S. Pat. No. 7,812,051 further discloses compositions comprising a carboxyl-containing polymer associated, via a linking agent of formula (III), with one or more quinone-containing therapeutic agents, wherein said linking agent of formula (III) is associated with said one or more quinone-containing therapeutic agents by a ketal linkage;

wherein said linking agent of formula (III) is of the form

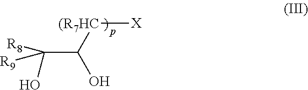

where

X is a hydroxyl, a protected hydroxyl or a protected amine;

each of R₇ and R₈ is independently selected from the group consisting of hydrogen, and (C₁-C₄) alkyl;

R₉ is H; and

p is 1-4.

U.S. Patent Publication No. 2006/0035963 discloses quinone prodrug compositions comprising a quinone compound covalently linked to one or two pro-moieties, such as an amino acid moiety or other water solubilizing moiety. The quinone compound may be released from the pro-moiety via hydrolytic, oxidative, and/or enzymatic release mechanisms. Preferred pro-moieties include amino acid moieties, such as amino acid residues, proteins and peptides; and carboxylic acids, such as malonic acid, succinic acid, and nicotinic acid.

The quinone prodrug compositions of the invention exhibit the added benefit of increased aqueous solubility, improved stability, and improved pharmacokinetic profiles. The pro-moiety may be selected to obtain desired prodrug characteristics. For example, the pro-moiety, e.g., an amino acid moiety or other water solubilizing moiety, may be selected based on solubility, stability, bioavailability, and/or in vivo delivery or uptake.

The pro-moiety may be attached at either or both of the quinone carbonyls. If a single pro-moiety is attached to the quinone compound, the uncomplexed quinone carbonyl group may independently be linked to a desired moiety to obtain desired properties. For instance, the uncomplexed quinone carbonyl may be substituted with a bioactive moiety to enhance the bioactivity of the quinone compound, or to confer an additional bioactivity to the quinone prodrug composition.

All stereoisomers of the compounds of the instant invention are contemplated, either in admixture or in pure or substantially pure form. The definition of the compounds according to the invention embraces all possible stereoisomers (e.g., the R and S configurations for each asymmetric center) and their mixtures. It very particularly embraces the racemic forms and the isolated optical isomers having a specified activity. The racemic forms can be resolved by physical methods, such as, for example, fractional crystallization, separation or crystallization of diastereomeric derivatives or separation by chiral column chromatography. The individual optical isomers can be obtained from the racemates by conventional methods, such as, for example, salt formation with an optically active acid followed by crystallization. Furthermore, all geometric isomers, such as E- and Z-configurations at a double bond, are within the scope of the invention unless otherwise stated. Certain compounds of this invention may exist in tautomeric forms. All such tautomeric forms of the compounds are considered to be within the scope of this invention unless otherwise stated.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have a common core structure, and are substituted with various groups as described herein. For example, all of the compounds represented by Formula I are pyrrolo-aminopyrimidine derivatives, and have Formula I as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

As used herein, the term “salt” is a pharmaceutically acceptable salt and can include acid addition salts including hydrochlorides, hydrobromides, phosphates, sulphates, hydrogen sulphates, alkylsulphonates, arylsulphonates, acetates, benzoates, citrates, maleates, fumarates, succinates, lactates, and tartrates; alkali metal cations such as Na, K, Li, alkali earth metal salts such as Mg or Ca, or organic amine salts.

The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.) the compounds of the present invention can be delivered in prodrug form. Thus, the present invention is intended to cover prodrugs of the presently claimed compounds, methods of delivering the same and compositions containing the same. Prodrugs are intended to include any covalently bonded carriers that release an active parent drug of the present invention in vivo when such prodrug is administered to a subject. Prodrugs of the present invention are prepared by modifying functional groups present in the compound in such a way that the modifications are cleaved, either in routine manipulation or in vivo, to the parent compound. Prodrugs include compounds of the present invention wherein a hydroxyl, amino, sulfhydryl, carboxy, or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetates, dialkylaminoacetates, formates, phosphates, sulfates, and benzoate derivatives) and carbamates (e.g., N,N-dimethylaminocarbonyl) of hydroxyl functional groups, esters (e.g. ethyl esters, morpholinoethanol esters) of carboxyl functional groups, N-acyl derivatives (e.g. N-acetyl), N-Mannich bases, Schiff bases and enaminones of amino functional groups, oximes, acetals, ketals and enol esters of ketone and aldehyde functional groups, and the like. See Bundegaard, H. “Design of Prodrugs” p 1-92, Elesevier, New York-Oxford (1985).

As used herein, the term “metabolite” means a product of metabolism of β-lapachone, or a pharmaceutically acceptable salt thereof that exhibits a similar activity in vivo to β-Lapachone.

Compounds of the present invention can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of compounds of the present invention.

Throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

The synthetic processes of the invention can tolerate a wide variety of functional groups, therefore various substituted starting materials can be used. The processes generally provide the desired final compound at or near the end of the overall process, although it may be desirable in certain instances to further convert the compound to a pharmaceutically acceptable salt, ester or prodrug thereof.

The present invention demonstrates that treatment with quinones, such as lapachone or an analog or derivative thereof or a pharmaceutically acceptable salt, prodrug, metabolite, polymorph or solvate thereof, strongly induced threonine phosphorylation of proteins of a MW of about 45 kD in cancer cells. One of the proteins was identified by mass spectrometry as NF45. The human amino acid sequence of NF45 is shown in Table 1 and identified as SEQ ID NO:1. Additional studies isolated NF45 protein phosphorlated at threonine 388 (T 388) in a TQ site (an ATM/ATR phosphorylation motif) near the carboxyl terminus. Thus, the present invention provides, in part, an isolated peptide comprising the amino acid sequence of SEQ ID NO: 1 phosphorylated at one or more serine, threonine, or tyrosine residues. Preferably, the isolated peptide is phosphorylated at one or more threonine residues. More preferably, the isolated peptide is phosphorylated at threonine residue T 388 of SEQ ID NO: 1. Most preferably, the isolated peptide is phosphorylated only at threonine residue T 388 and not phosphorylated at any other amino acid residues.

Phosphorylation is one of several post-translational mechanisms by which the biological activity of transcription factors can be regulated. In the absence of phospho-specific antibodies, in vivo analysis of phosphorylation requires the use of large amounts of radioactivity for the metabolic labeling of the cells followed by extensive manipulation of the resulting radioactive total cell extracts. The resulting radioactive phospho-analysis cannot differentiate different sites on the same protein, nor can it identify the exact site of phosphorylation on the protein. Antibodies that can recognize a phosphorylation event at a specific amino acid negate the need for large amounts of radioactivity and allow the researcher to very easily follow the status of phosphorylation at a specific amino acid throughout various biological processes.

To further ascertain the phosphorylation status of NF45 following treatment with quinones, such as lapachone or an analog or derivative thereof or a pharmaceutically acceptable salt, prodrug, metabolite, polymorph or solvate thereof, a custom antibody against human pNF45 (T388) was synthesized. Thus, the present invention also provides, in part, an isolated antibody or antigen-binding portion thereof that specifically binds a protein comprising the amino acid sequence of SEQ ID NO: 1 phosphorylated at one or more serine, threonine, or tyrosine residues. Preferably, the isolated antibody specifically binds a protein of SEQ ID NO: 1 when phosphorylated at a threonine residue. More preferably, the isolated antibody specifically binds a protein of SEQ ID NO: 1 when phosphorylated at threonine residue T 388. Most preferably, the isolated antibody only binds the protein when phosphorylated at threonine residue T 388 and not phosphorylated at any other amino acid residues. The antibody can be monoclonal, polyclonal, humanized, fully-human or chimeric. The isolated phosphorylation site-specific antibody or antigen-binding portion thereof, was generated utilizing a peptide corresponding to amino acids 383 to 390 (EESME-T-QE) (SEQ ID NO: 2) of human NF45 which was phosphorylated at threonine 388. Thus, the present invention also provides an isolated peptide comprising, consisting essential of, or consisting of, the amino acid sequence of SED ID NO: 2 phosphorylated at threonine residue T 6.

The present invention also provides, in part, methods for producing an antibody of the invention. One method includes: (a) introducing an isolated peptide of the invention to a mammal in an amount sufficient to produce an antibody to said peptide; and (b) recovering said antibody from said mammal. Another method includes: (a) introducing an isolated peptide of the invention to a mammal in an amount sufficient to elicit lymphocytes that are capable of producing an antibody to said peptide; (b) recovering said lymphocytes from said mammal; (c) fusing said lymphocytes with myeloma cells; (d) culturing said hybridoma cells under appropriate growth conditions; and (e) recovering one or more monoclonal antibodies to said peptide from the cell culture media The method can further comprise purifying the one or more monoclonal antibodies recovered in step (e). Preferably the isolated peptide comprises the amino acid sequence of SEQ ID NO: 1 phosphorylated at one or more serine, threonine, or tyrosine residues. More preferably, the isolated peptide is phosphorylated at one or more threonine residues. Most preferably, the isolated peptide is phosphorylated at threonine residue T 388 of SEQ ID NO: 1 or an isolated peptide comprising, consisting essential of, or consisting of, the amino acid sequence of SED ID NO: 2 phosphorylated at threonine residue T 6.

The term “antibody” as used herein includes all forms of antibodies, including but not limited to recombinant antibodies, chimeric antibodies, single chain antibodies, humanized antibodies, monoclonal antibodies, polyclonal antibodies, non-human antibodies, and fully human antibodies. Antibodies can be of any isotype including IgM, IgG, IgD, IgA and IgE, and any sub-Isotype, including IgG1, IgG2a, IgG2b, IgG3 and IgG4, IgE1, IgE2, etc. The light chains of the antibodies can either be kappa light chains or lambda light chains. The modifier “monoclonal” indicates the character of the antibody as being obtained from a substantially homogeneous population of antibodies, and is not to be construed as requiring production of the antibody by any particular method. For example, monoclonal antibodies useful for methods described herein be made by the hybridoma method first described by Kohler et al., Nature, 256:495 (1975), or can be made by recombinant DNA methods (see, e.g., U.S. Pat. No. 4,816,567). The “monoclonal antibodies” can also be isolated from phage antibody libraries using the techniques described in Clackson et al., Nature, 352:624-628 (1991) and Marks et al., J. Mol. Biol., 222:581-597 (1991) Chimeric antibodies include those antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)).

Methods for generating antibodies against an antigen of interest are known in the art. For example, polyclonal antibodies can be raised in animals by multiple subcutaneous (sc) or intraperitoneal (ip) injections of the relevant antigen and an adjuvant. It can be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl2, or R1N═C═NR, where R and R1 are different alkyl groups. Animals can be immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals can be boosted with one fifth to one tenth of the original amount of peptide or conjugate in Freund's complete adjuvant by subcutaneous injection at multiple sites. Seven to 14 days later the animals can be bled and the serum is assayed for antibody titer. Animals can be boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Methods for producing monoclonal antibodies are known in the art, and include, for example, the hybridoma method. In the hybridoma method, a mouse or other appropriate host animal, such as a hamster or rabbit, is immunized as hereinabove described to elicit lymphocytes that produce or are capable of producing antibodies that will specifically bind to the protein used for immunization. Alternatively, lymphocytes can be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT or other similar marker gene), the culture medium for the hybridomas typically will include hypoxanthine, aminopterin, and thymidine (HAT medium), which substances prevent the growth of HGPRT-deficient cells. Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59-103 (Academic Press, 1986)). The monoclonal antibodies secreted by the subclones are suitably separated from the culture medium, ascites fluid, or serum by conventional antibody purification procedures such as, for example, protein A-Sepharose, hydroxylapatite chromatography, gel electrophoresis, dialysis, or affinity chromatography. DNA encoding the monoclonal antibodies is readily isolated and sequenced using conventional procedures (e.g., by using oligonucleotide probes that are capable of binding specifically to genes encoding the heavy and light chains of murine antibodies along with techniques such as PCR). The hybridoma cells serve as a preferred source of such DNA. Once isolated, the DNA can be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Methods for further manipulating antibodies based on sequence modification, chimerization, humanization, structural modeling, and fragmentation are known in the art.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J H) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807. Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M113 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597-(1991), or Griffith et al., EMBO J. 12:725-734 (1993). See, also, U.S. Pat. Nos. 5,565,332 and 5,573,905. Human antibodies can also be generated by in vitro activated B cells (see U.S. Pat. Nos. 5,567,610 and 5,229,275).

“Antibody fragments” include any portion of an intact antibody, preferably comprising the antigen-binding or variable region thereof. Non-limiting examples of antibody fragments include Fab, Fab′, F(ab′)₂, and Fv fragments; diabodies; linear antibodies; single-chain antibody molecules; and multispecific antibodies formed from antibody fragment(s). Humanized forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, avian, other mammalian or non-mammalian animals or nonhuman primate having the desired specificity, affinity, and capacity. In some instances, framework region (FR) residues of the human immunoglobulin are replaced by corresponding non-human residues. Furthermore, humanized antibodies can comprise residues that are not found in the recipient antibody or in the donor antibody. These modifications can be made to further refine antibody performance. For further details on humanized antibodies, see Jones et al., Nature 321:522-525 (1986); Riechmann et al., Nature 332:323-329 (1988); and Presta, Curr. Op. Struct. Biol. 2:593-596 (1992).

Various techniques have been developed for the production of antibody fragments comprising one or more antigen binding regions. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 (1992)). According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment can also be a “linear antibody”, e.g., as described in U.S. Pat. No. 5,641,870 for example. Such linear antibody fragments can be monospecific or bispecific.

As used herein, “polyclonal antibody” refers to an antibody obtained from a mixture of immunoglobulin molecules secreted against a specific antigen, each recognizing a different epitope.

As used herein, the phrase “oligoclonal antibodies” refers to a predetermined mixture of distinct monoclonal antibodies. See, e.g., PCT publication WO 95/20401; U.S. Pat. Nos. 5,789,208 and 6,335,163.

The term “antigen-binding fragment” is used to include fragments of the antibody that still bind the antigen. The term “epitope” herein used is meant to refer to that portion of any molecule capable of being bound by an antibody which can also be recognized by that antibody.

The term “phospho-specific antibody” herein used means a specific antibody against the phosphorylated amino acid residue. A phospho-specific antibody does not detect un-phosphorylated residues.

As used herein, “peptide” includes all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. Routine experimentation will determine whether a mimetic is within the scope of the invention, i.e., that its structure and/or function is not substantially altered.

As used herein, “polypeptide” includes a molecular chain of amino acids linked through peptide bonds. “Polypeptide” does not refer to a specific length of the product. Thus, “peptides,” “oligopeptides,” and “proteins” are included within the definition of polypeptide.

As used herein, an “antigen”, includes any portion of a protein (peptide, protein fragment, full-length protein), wherein the protein is naturally occurring or synthetically derived, a cellular composition (whole cell, cell lysate or disrupted cells), an organism (whole organism, lysate or disrupted cells), a carbohydrate, a lipid, or other molecule, or a portion thereof, wherein the antigen elicits an antigen-specific immune response (humoral and/or cellular immune response).

As used herein, the term “specifically binds” refers to the binding specificity of a specific binding pair. Recognition by an antibody of a particular target in the presence of other potential targets is one characteristic of such binding. “Binding component member” refers to a member of a specific binding pair, i.e., two different molecules wherein one of the molecules specifically binds with the second molecule through chemical or physical means. The two molecules are related in the sense that their binding with each other is such that they are capable of distinguishing their binding partner from other assay constituents having similar characteristics. The members of the binding component pair are referred to as ligand and receptor (antiligand), specific binding pair (sbp) member and sbp partner, and the like. A molecule may also be a sbp member for an aggregation of molecules; for example an antibody raised against an immune complex of a second antibody and its corresponding antigen may be considered to be an sbp member for the immune complex.

As used herein the term “does not specifically bind” refers to the specificity of particular antibodies or antibody fragments. Antibodies or antibody fragments that do not specifically bind a particular moiety generally contain a specificity such that a large percentage of the particular moiety would not be bound by such antibodies or antibody fragments. This percentage generally lies within the acceptable cross reactivity percentage with interfering moieties of assays utilizing antibodies directed to detecting a specific target. Frequently, antibodies or antibody fragments of the present disclosure do not specifically bind greater than about 90% of an interfering moiety, although higher percentages are clearly contemplated and preferred. For example, antibodies or antibody fragments of the present disclosure do not specifically bind about 91%, about 92%, about 93%, about 94%, about 95%, about 96%, about 97%, about 98%, about 99%, and about 99% or more of an interfering moiety. Less occasionally, antibodies or antibody fragments of the present disclosure do not specifically bind greater than about 70%, or greater than about 75%, or greater than about 80%, or greater than about 85% of an interfering moiety.

The studies described in detail in the Examples shows that quinones, such as lapachone or an analog or derivative thereof or a pharmaceutically acceptable salt, prodrug, metabolite, polymorph or solvate thereof increased pNF45 (T388) in a time- and concentration-dependent manner accompanied by activation of ATM and DNA-PK. Inhibition of ATM and DNA-PK suppress lapachone-induced pNF45 (T388) elevation.

Evidence that lapachone induces DNA damage is supported by the observation of CHK2 phosphorylation and g-H2AX elevation in treated cells. As such, the effects of other DNA damaging agents on NF45 phosphorylation were investigated and it was shown that lapachone was able to increase pNF45 (T388) levels in a similar way as topoisomerase inhibitors (doxorubicin and camptothecin) but not a DNA-crosslinking agent (cisplatin). Thus, a candidate compound of the present invention can be any quinone, such as, lapachone or an analog or derivative thereof or a pharmaceutically acceptable salt, prodrug, metabolite, polymorph or solvate thereof.

Topoisomerase inhibitors are agents designed to interfere with the action of topoisomerase enzyme (topoisomerase I and II), which are enzymes that control the changes in DNA structure by catalyzing the breaking and rejoining of the phosphodiester backbone of DNA strands during the normal cell cycle. Exemplary topoisomerase poison drugs include, but are not limited to, teniposide, etoposide, adriamycin, camptothecin, daunorubicin, dactinomycin, mitoxantrone, amsacrine, epirubicin and idarubicin.

Immunofluorescence staining of β-Lapachone treated cells using pNF45 (T388) antibody showed a significant increase in pNF45 (T388) levels predominantly in nuclei. These studies demonstrate that phosphorylation of NF45 at threonine 388 can serve as a pharmacodynamic (PD) biomarker.

Therefore, the present invention also provides, in part, a method comprising (a) exposing a subject to a candidate compound; (b) obtaining a biological sample from said subject following said exposure; (c) determining the presence, absence, or amount of a peptide comprising the amino acid sequence of SEQ ID NO:1 phosphorylated at one or more serine, threonine, or tyrosine residues in said biological sample; and, (d) comparing the presence, absence, or amount of said peptide in said biological sample with the presence, absence, or amount of said peptide determined in a biological sample obtained from a subject not exposed to the candidate compound. Determining the presence, absence, or amount of a peptide comprising the amino acid sequence of SEQ ID NO:1 phosphorylated at one or more serine, threonine, or tyrosine residues in said biological sample can include contacting said biological sample with an isolated antibody or antigen-binding portion thereof that specifically binds to said peptide comprising the amino acid sequence of SEQ ID NO:1 phosphorylated at one or more serine, threonine, or tyrosine residues and assessing a complex formed between said peptide and said antibody. The antibody can specifically bind to the peptide phosphorylated at one or more threonine residues. More preferably, the antibody specifically binds to the peptide phosphorylated at threonine residue T388. The detection step can include flow cytometry, an immunohistochemical assay, ELISA or a Western Blot. The sample can be any biological sample. Preferably the sample is a cell or tissue sample. The subject can be a mammal. Preferably, the subject is a human.

The present invention also provides, in part, a method including (a) contacting a biological sample with an isolated antibody provided by the instant invention and described herein; and, (b) assessing a complex formed between a peptide comprising the amino acid sequence of SEQ ID NO:1 phosphorylated at one or more serine, threonine, or tyrosine residues and said antibody to determine the presence, absence, or amount of said peptide in said biological sample.

The present invention also provides, in part, a method comprising (a) contacting a biological sample with a candidate compound; (b) determining the presence, absence, or amount of a peptide comprising the amino acid sequence of SEQ ID NO:1 phosphorylated at one or more serine, threonine, or tyrosine residues in said biological sample following contact with the candidate compound; and, (c) comparing the presence, absence, or amount of said peptide in said biological sample with the presence, absence, or amount of said peptide determined in a biological sample not contacted with the candidate compound.

The methods of the present invention further include, in part, identifying a candidate compound. The candidate compound can be a compound capable of inducing or increasing the presence of a peptide comprising the amino acid sequence of SEQ ID NO:1 phosphorylated at one or more serine, threonine, or tyrosine residues in the subject or sample. Inducing or increasing the presence of a peptide comprising the amino acid sequence of SEQ ID NO:1 phosphorylated at one or more serine, threonine, or tyrosine residues includes inducing or increasing the phosphorylation state or level of a peptide comprising the amino acid sequence of SEQ ID NO: 1, when compared to the absence of the candidate compound.

The candidate compound of the present invention can be a topoisomerase inhibitor. More preferably, a topoisomerase I inhibitor. The candidate compound of the present invention can be a quinone, naptho-quinone or ortho-quinone. The candidate compound is a lapachone or an analog or derivative thereof or a pharmaceutically acceptable salt, prodrug, metabolite, polymorph or solvate thereof. Preferably, the candidate compound is β-Lapachone or an analog or derivative thereof or (S)-1′-(3-(4-(tert-butyl)phenoxy)-2-hydroxypropyl)-2H-spiro[naphtho[1,2-b][1,4]oxathiine-3,4′-piperidine]-5,6-dione or an analog or derivative thereof. More preferably, the candidate compound is a prodrug of β-Lapachone. Most preferably, the candidate compound is a

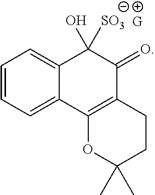

As used herein the term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregate of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

The methods of the present invention further include, in part, identifying a subject having an induction or increase in a peptide comprising the amino acid sequence of SEQ ID NO:1 phosphorylated at one or more serine, threonine, or tyrosine residues following exposure to a candidate compound and administering a chemotherapeutic agent or radiation therapy to said subject. The subject can be a mammal. Preferably, the subject is a human. The subject can be suffering from, or diagnosed with, a cell proliferation disorder. The cell proliferation disorder can be cancer or pre-cancer. Preferably, the administration of the candidate compound, the chemotherapeutic agent or the radiation therapy, alone or in any combination thereof, treats the proliferation disorder in said subject

The present invention also provides, in part, a method for inducing or increasing the amount of a peptide comprising the amino acid sequence of SEQ ID NO:1 phosphorylated at one or more serine, threonine, or tyrosine residues in a hyper-proliferating cell comprising contacting the cell with candidate compound. Preferably, the isolated peptide is phosphorylated at one or more threonine residues. More preferably, the isolated peptide is phosphorylated at threonine residue T 388 of SEQ ID NO: 1. Most preferably, the isolated peptide is phosphorylated only at threonine residue T 388 and not phosphorylated at any other amino acid residues. The cell can be in vivo, in vitro or ex vivo. The cell can be a cancer cell or pre-cancer cell.

As used herein, a “subject in need thereof” is a subject having a cell proliferative disorder, or a subject having an increased risk of developing a cell proliferative disorder relative to the population at large. A subject in need thereof can have a precancerous condition. Preferably, a subject in need thereof has cancer. A “subject” includes a mammal. The mammal can be e.g., any mammal, e.g., a human, primate, bird, mouse, rat, fowl, dog, cat, cow, horse, goat, camel, sheep or a pig. Preferably, the mammal is a human.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the invention encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. Preferably, the methods provided herein are used to treat or alleviate a symptom of cancer. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; and diabetes mellitus.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, uringary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A “cell proliferative disorder of the hematologic system” is a cell proliferative disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. Preferably, compositions of the present invention may be used to treat a cancer selected from the group consisting of a hematologic cancer of the present invention or a hematologic cell proliferative disorder of the present invention. A hematologic cancer of the present invention can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “cell proliferative disorder of the lung” is a cell proliferative disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Preferably, compositions of the present invention may be used to treat lung cancer or cell proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma”, bronchioalveolar carcinoma, giant cell carcinoma, spindle cell carcinoma, and large cell neuroendocrine carcinoma. Lung cancer can include lung neoplasms having histologic and ultrastructual heterogeneity (e.g., mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A “cell proliferative disorder of the colon” is a cell proliferative disorder involving cells of the colon. Preferably, the cell proliferative disorder of the colon is colon cancer. Preferably, compositions of the present invention may be used to treat colon cancer or cell proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Cell proliferative disorders of the colon can include all forms of cell proliferative disorders affecting colon cells. Cell proliferative disorders of the colon can include colon cancer, precancerous conditions of the colon, adenomatous polyps of the colon and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A “cell proliferative disorder of the pancreas” is a cell proliferative disorder involving cells of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysaplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructual heterogeneity (e.g., mixed cell types).

A “cell proliferative disorder of the prostate” is a cell proliferative disorder involving cells of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate can include hyperplasia, metaplasia, and dysplasia of the prostate.

A “cell proliferative disorder of the skin” is a cell proliferative disorder involving cells of the skin Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A “cell proliferative disorder of the ovary” is a cell proliferative disorder involving cells of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the skin can include hyperplasia, metaplasia, and dysplasia of cells of the ovary.

A “cell proliferative disorder of the breast” is a cell proliferative disorder involving cells of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

A cell proliferative disorder of the breast can be a precancerous condition of the breast. Compositions of the present invention may be used to treat a precancerous condition of the breast. A precancerous condition of the breast can include atypical hyperplasia of the breast, ductal carcinoma in situ (DCIS), intraductal carcinoma, lobular carcinoma in situ (LCIS), lobular neoplasia, and stage 0 or grade 0 growth or lesion of the breast (e.g., stage 0 or grade 0 breast cancer, or carcinoma in situ). A precancerous condition of the breast can be staged according to the TNM classification scheme as accepted by the American Joint Committee on Cancer (AJCC), where the primary tumor (T) has been assigned a stage of T0 or T is; and where the regional lymph nodes (N) have been assigned a stage of N0; and where distant metastasis (M) has been assigned a stage of M0.

The cell proliferative disorder of the breast can be breast cancer. Preferably, compositions of the present invention may be used to treat breast cancer. Breast cancer includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphcytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructual heterogeneity (e.g., mixed cell types).

Preferably, compound of the present invention, or a pharmaceutically acceptable salt, prodrug, metabolite, polymorph or solvate thereof, may be used to treat breast cancer. A breast cancer that is to be treated can include familial breast cancer. A breast cancer that is to be treated can include sporadic breast cancer. A breast cancer that is to be treated can arise in a male subject. A breast cancer that is to be treated can arise in a female subject. A breast cancer that is to be treated can arise in a premenopausal female subject or a postmenopausal female subject. A breast cancer that is to be treated can arise in a subject equal to or older than 30 years old, or a subject younger than 30 years old. A breast cancer that is to be treated has arisen in a subject equal to or older than 50 years old, or a subject younger than 50 years old. A breast cancer that is to be treated can arise in a subject equal to or older than 70 years old, or a subject younger than 70 years old.

A breast cancer that is to be treated can be typed to identify a familial or spontaneous mutation in BRCA1, BRCA2, or p53. A breast cancer that is to be treated can be typed as having a HER2/neu gene amplification, as overexpressing HER2/neu, or as having a low, intermediate or high level of HER2/neu expression. A breast cancer that is to be treated can be typed for a marker selected from the group consisting of estrogen receptor (ER), progesterone receptor (PR), human epidermal growth factor receptor-2, Ki-67, CA15-3, CA 27-29, and c-Met. A breast cancer that is to be treated can be typed as ER-unknown, ER-rich or ER-poor. A breast cancer that is to be treated can be typed as ER-negative or ER-positive. ER-typing of a breast cancer may be performed by any reproducible means. ER-typing of a breast cancer may be performed as set forth in Onkologie 27: 175-179 (2004). A breast cancer that is to be treated can be typed as PR-unknown, PR-rich or PR-poor. A breast cancer that is to be treated can be typed as PR-negative or PR-positive. A breast cancer that is to be treated can be typed as receptor positive or receptor negative. A breast cancer that is to be treated can be typed as being associated with elevated blood levels of CA 15-3, or CA 27-29, or both.

A breast cancer that is to be treated can include a localized tumor of the breast. A breast cancer that is to be treated can include a tumor of the breast that is associated with a negative sentinel lymph node (SLN) biopsy. A breast cancer that is to be treated can include a tumor of the breast that is associated with a positive sentinel lymph node (SLN) biopsy. A breast cancer that is to be treated can include a tumor of the breast that is associated with one or more positive axillary lymph nodes, where the axillary lymph nodes have been staged by any applicable method. A breast cancer that is to be treated can include a tumor of the breast that has been typed as having nodal negative status (e.g., node-negative) or nodal positive status (e.g., node-positive). A breast cancer that is to be treated can include a tumor of the breast that has metastasized to other locations in the body. A breast cancer that is to be treated can be classified as having metastasized to a location selected from the group consisting of bone, lung, liver, or brain. A breast cancer that is to be treated can be classified according to a characteristic selected from the group consisting of metastatic, localized, regional, local-regional, locally advanced, distant, multicentric, bilateral, ipsilateral, contralateral, newly diagnosed, recurrent, and inoperable.

A compound of the present invention, or a pharmaceutically acceptable salt, prodrug, metabolite, polymorph or solvate thereof, when administered in an amount sufficient to induce or increase the phosphorylation levels of NF45 protein within the cells of said patient may be used to treat or prevent a cell proliferative disorder of the breast, or to treat or prevent breast cancer, in a subject having an increased risk of developing breast cancer relative to the population at large. A subject with an increased risk of developing breast cancer relative to the population at large is a female subject with a family history or personal history of breast cancer. A subject with an increased risk of developing breast cancer relative to the population at large is a female subject having a germ-line or spontaneous mutation in BRCA1 or BRCA2, or both. A subject with an increased risk of developing breast cancer relative to the population at large is a female subject with a family history of breast cancer and a germ-line or spontaneous mutation in BRCA1 or BRCA2, or both. A subject with an increased risk of developing breast cancer relative to the population at large is a female who is greater than 30 years old, greater than 40 years old, greater than 50 years old, greater than 60 years old, greater than 70 years old, greater than 80 years old, or greater than 90 years old. A subject with an increased risk of developing breast cancer relative to the population at large is a subject with atypical hyperplasia of the breast, ductal carcinoma in situ (DCIS), intraductal carcinoma, lobular carcinoma in situ (LCIS), lobular neoplasia, or a stage 0 growth or lesion of the breast (e.g., stage 0 or grade 0 breast cancer, or carcinoma in situ).

A breast cancer that is to be treated can histologically graded according to the Scarff-Bloom-Richardson system, wherein a breast tumor has been assigned a mitosis count score of 1, 2, or 3; a nuclear pleiomorphism score of 1, 2, or 3; a tubule formation score of 1, 2, or 3; and a total Scarff-Bloom-Richardson score of between 3 and 9. A breast cancer that is to be treated can be assigned a tumor grade according to the International Consensus Panel on the Treatment of Breast Cancer selected from the group consisting of grade 1, grade 1-2, grade 2, grade 2-3, or grade 3.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1(mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “candidate compound” refers to a compound of the present invention, or a pharmaceutically acceptable salt, prodrug, metabolite, polymorph or solvate thereof, that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the present invention, or a pharmaceutically acceptable salt, prodrug, metabolite, polymorph or solvate thereof. The biological or medical response can be the treatment of cancer. The biological or medical response can be treatment or prevention of a cell proliferative disorder. In vitro or in vivo biological assays can include, but are not limited to, enzymatic activity assays, electrophoretic mobility shift assays, reporter gene assays, in vitro cell viability assays, and the assays described herein.

As used herein, “monotherapy” refers to the administration of a single active or therapeutic compound to a subject in need thereof. Preferably, monotherapy will involve administration of a therapeutically effective amount of an active compound. For example, cancer monotherapy with one of the compound of the present invention, or a pharmaceutically acceptable salt, prodrug, metabolite, analog or derivative thereof, to a subject in need of treatment of cancer. Monotherapy may be contrasted with combination therapy, in which a combination of multiple active compounds is administered, preferably with each component of the combination present in a therapeutically effective amount. In one aspect, monotherapy with a compound of the present invention, or a pharmaceutically acceptable salt, prodrug, metabolite, polymorph or solvate thereof, is more effective than combination therapy in inducing a desired biological effect.

As used herein, “treating” or “treat” describes the management and care of a patient for the purpose of combating a disease, condition, or disorder and includes the administration of a compound of the present invention, or a pharmaceutically acceptable salt, prodrug, metabolite, polymorph or solvate thereof, to alleviate the symptoms or complications of a disease, condition or disorder, or to eliminate the disease, condition or disorder.

A compound of the present invention, or a pharmaceutically acceptable salt, prodrug, metabolite, polymorph or solvate thereof, can also be administered in amount sufficient to induce or increase the phosphorylation levels of NF45 protein within the cells of said patient used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the invention, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

But sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage.

A cancer may also cause symptoms such as fever, fatigue, or weight loss. This may be because cancer cells use up much of the body's energy supply or release substances that change the body's metabolism. Or the cancer may cause the immune system to react in ways that produce these symptoms.

Sometimes, cancer cells release substances into the bloodstream that cause symptoms not usually thought to result from cancers. For example, some cancers of the pancreas can release substances which cause blood clots to develop in veins of the legs. Some lung cancers make hormone-like substances that affect blood calcium levels, affecting nerves and muscles and causing weakness and dizziness

Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma.

Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease.

Along with cancers of the skin (see next section), some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the instant invention.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, prodrug, metabolite, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt, prodrug, metabolite, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

As used herein, the term “selectively” means tending to occur at a higher frequency in one population than in another population. The compared populations can be cell populations. Preferably, a compound of the present invention, or a pharmaceutically acceptable salt, prodrug, metabolite, polymorph or solvate thereof, acts selectively on a cancer or precancerous cell but not on a normal cell. Preferably, a compound of the present invention, or a pharmaceutically acceptable salt, prodrug, metabolite, polymorph or solvate thereof, acts selectively to modulate one molecular target (e.g., a target kinase) but does not significantly modulate another molecular target (e.g., a non-target kinase). The invention also provides a method for selectively inhibiting the activity of an enzyme, such as a kinase. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

A compound of the present invention, or a pharmaceutically acceptable salt, prodrug, metabolite, polymorph or solvate thereof, can modulate the activity of a molecular target (e.g., a target kinase). Modulating refers to stimulating or inhibiting an activity of a molecular target. Preferably, a compound of the present invention, or a pharmaceutically acceptable salt, prodrug, metabolite, polymorph or solvate thereof, modulates the activity of a molecular target if it stimulates or inhibits the activity of the molecular target by at least 2-fold relative to the activity of the molecular target under the same conditions but lacking only the presence of said compound. More preferably, a compound of the present invention, or a pharmaceutically acceptable salt, prodrug, metabolite, polymorph or solvate thereof, modulates the activity of a molecular target if it stimulates or inhibits the activity of the molecular target by at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro by an enzymatic activity assay or a DNA binding assay, or the activity of a molecular target may be measured in vivo by assaying for expression of a reporter gene. Preferably, the molecular target to be modulated is NF45. More preferably, NF45 is modulated by inducing or increasing the phosphorylation status or level of NF45. Most preferably, NF45 is phosphorlated on the Threonine at amino acid residue 388 of SEQ ID NO: 1.

A compound of the present invention, or a pharmaceutically acceptable salt, prodrug, metabolite, polymorph or solvate thereof, does not significantly modulate the activity of a molecular target if the addition of the compound does not stimulate or inhibit the activity of the molecular target by greater than 10% relative to the activity of the molecular target under the same conditions but lacking only the presence of said compound.

As used herein, the term “isozyme selective” means preferential inhibition or stimulation of a first isoform of an enzyme in comparison to a second isoform of an enzyme (e.g., preferential inhibition or stimulation of a kinase isozyme alpha in comparison to a kinase isozyme beta). Preferably, a compound of the present invention, or a pharmaceutically acceptable salt, prodrug, metabolite, polymorph or solvate thereof, demonstrates a minimum of a four fold differential, preferably a ten fold differential, more preferably a fifty fold differential, in the dosage required to achieve a biological effect. Preferably, a compound of the present invention, or a pharmaceutically acceptable salt, prodrug, metabolite, polymorph or solvate thereof, demonstrates this differential across the range of inhibition, and the differential is exemplified at the IC₅₀, i.e., a 50% inhibition, for a molecular target of interest.

Administering a compound of the present invention, or a pharmaceutically acceptable salt, prodrug, metabolite, polymorph or solvate thereof, to a cell or a subject in need thereof can result in modulation (i.e., stimulation or inhibition) of an activity of a kinase of interest.

The present invention provides methods to assess biological activity of a compound of the present invention, or a pharmaceutically acceptable salt, prodrug, metabolite, polymorph or solvate thereof. In one method, an assay based on enzymatic activity can be utilized. In one specific enzymatic activity assay, the enzymatic activity is from a kinase. As used herein, “kinase” refers to a large class of enzymes which catalyze the transfer of the γ-phosphate from ATP to the hydroxyl group on the side chain of Ser/Thr or Tyr in proteins and peptides and are intimately involved in the control of various important cell functions, perhaps most notably: signal transduction, differentiation, and proliferation. There are estimated to be about 2,000 distinct protein kinases in the human body, and although each of these phosphorylates particular protein/peptide substrates, they all bind the same second substrate ATP in a highly conserved pocket. About 50% of the known oncogene products are protein tyrosine kinases (PTKs), and their kinase activity has been shown to lead to cell transformation. Preferably, the kinase assayed is a tyrosine kinase.

A change in enzymatic activity caused by a compound of the present invention, or a pharmaceutically acceptable salt, prodrug, metabolite, polymorph or solvate thereof, can be measured in the disclosed assays. The change in enzymatic activity can be characterized by the change in the extent of phosphorylation of certain substrates. As used herein, “phosphorylation” refers to the addition of phosphate groups to a substrate, including proteins and organic molecules; and, plays an important role in regulating the biological activities of proteins. Preferably, the phosphorylation assayed and measured involves the addition of phosphate groups to tyrosine residues. The substrate can be a peptide or protein.

In some assays, immunological reagents, e.g., antibodies and antigens, are employed. Fluorescence can be utilized in the measurement of enzymatic activity in some assays. As used herein, “fluorescence” refers to a process through which a molecule emits a photon as a result of absorbing an incoming photon of higher energy by the same molecule. Specific methods for assessing the biological activity of the disclosed compounds are described in the examples.

Administering a compound of the present invention, or a pharmaceutically acceptable salt, prodrug, metabolite, polymorph or solvate thereof, to a cell or a subject in need thereof results in modulation (i.e., stimulation or inhibition) of an activity of an intracellular target (e.g., substrate). Preferably, the intracellular target is a target which induces or increases the phorsphorylation status/level of NF45.

Activating refers to placing a composition of matter (e.g., protein or nucleic acid) in a state suitable for carrying out a desired biological function. A composition of matter capable of being activated also has an unactivated state. An activated composition of matter may have an inhibitory or stimulatory biological function, or both.

Elevation refers to an increase in a desired biological activity of a composition of matter (e.g., a protein or a nucleic acid). Elevation may occur through an increase in concentration of a composition of matter.

As used herein, “a cell cycle checkpoint pathway” refers to a biochemical pathway that is involved in modulation of a cell cycle checkpoint. A cell cycle checkpoint pathway may have stimulatory or inhibitory effects, or both, on one or more functions comprising a cell cycle checkpoint. A cell cycle checkpoint pathway is comprised of at least two compositions of matter, preferably proteins, both of which contribute to modulation of a cell cycle checkpoint. A cell cycle checkpoint pathway may be activated through an activation of one or more members of the cell cycle checkpoint pathway. Preferably, a cell cycle checkpoint pathway is a biochemical signaling pathway.

As used herein, “cell cycle checkpoint regulator” refers to a composition of matter that can function, at least in part, in modulation of a cell cycle checkpoint. A cell cycle checkpoint regulator may have stimulatory or inhibitory effects, or both, on one or more functions comprising a cell cycle checkpoint. A cell cycle checkpoint regulator can be a protein or not a protein.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, cell death occurs by apoptosis.

Preferably, an effective amount of a compound of the present invention, or a pharmaceutically acceptable salt, prodrug, metabolite, polymorph or solvate thereof, is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a compound of the present invention, or a pharmaceutically acceptable salt, prodrug, metabolite, polymorph or solvate thereof, can induce or activate cell death selectively in cancer cells. Administering to a subject in need thereof a compound of the present invention, or a pharmaceutically acceptable salt, prodrug, metabolite, polymorph or solvate thereof, can induce or activate cell death selectively in cancer cells. Contacting a cell with a compound of the present invention, or a pharmaceutically acceptable salt, prodrug, metabolite, polymorph or solvate thereof, can induce cell death selectively in one or more cells affected by a cell proliferative disorder. Preferably, administering to a subject in need thereof a compound of the present invention, or a pharmaceutically acceptable salt, prodrug, metabolite, polymorph or solvate thereof, induces cell death selectively in one or more cells affected by a cell proliferative disorder.

The present invention relates to a method of treating or preventing cancer by administering a compound of the present invention, or a pharmaceutically acceptable salt, prodrug, metabolite, polymorph or solvate thereof, to a subject in need thereof, where administration of the compound of the present invention, or a pharmaceutically acceptable salt, prodrug, metabolite, polymorph or solvate thereof, results in one or more of the following: accumulation of cells in G1 and/or S phase of the cell cycle, cytotoxicity via cell death in cancer cells without a significant amount of cell death in normal cells, antitumor activity in animals with a therapeutic index of at least 2, and activation of a cell cycle checkpoint. As used herein, “therapeutic index” is the maximum tolerated dose divided by the efficacious dose.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the invention

As used herein, “combination therapy” or “co-therapy” includes the administration of a compound of the present invention, or a pharmaceutically acceptable salt, prodrug, metabolite, polymorph or solvate thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may be, but generally is not, intended to encompass the administration of two or more of these therapeutic agents as part of separate monotherapy regimens that incidentally and arbitrarily result in the combinations of the present invention.

“Combination therapy” is intended to embrace administration of these therapeutic agents in a sequential manner, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

“Combination therapy” also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

A compound of the present invention, or a pharmaceutically acceptable salt, prodrug, metabolite, analog or derivative thereof, may be administered in combination with a second chemotherapeutic agent. The second chemotherapeutic agent (also referred to as an anti-neoplastic agent or anti-proliferative agent) can be an alkylating agent; an antibiotic; an anti-metabolite; a detoxifying agent; an interferon; a polyclonal or monoclonal antibody; an EGFR inhibitor, a HER2 inhibitor; a histone deacetylase inhibitor; a hormone; a mitotic inhibitor, an MTOR inhibitor; a multi-kinase inhibitor, a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase inhibitor), a cytidine analogue drug or any chemotherapeutic, anti-neoplastic or anti-proliferative agent listed in www.cancer.org/docroot/cdg/cdg_{—}0.asp.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodine¹³¹ tositumomab (Bexxar); alemtuzumab (Campath); ibritumomab (Zevalin; In-111; Y-90 Zevalin); gemtuzumab (Mylotarg); eculizumab (Soliris) ordenosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569.

Exemplary HER2 inhibitors include, but are not limited to, trastuzumab (Herceptin); lapatinib (Tykerb) or AC-480.

Histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus (Torisel); rapamune, ridaforolimus; or AP23573.

Exemplary multi-kinase inhibitors include, but are not limited to, sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/easudil hydrochloride; flavopiridol; seliciclib (CYC202; Roscovitrine); SNS-032 (BMS-387032); Pkc412; bryostatin; KAI-9803; SF1126; VX-680; Azd1152; Arry-142886 (AZD-6244); SCIO-469; GW681323; CC-401; CEP-1347 or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib.

Exemplary microtubule targeting drugs include, but are not limited to, paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexylen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur—0.4 M 5-chloro-2,4-dihydroxypyrimidine—1 M potassium oxonate) or lovastatin.

In another aspect, the second chemotherapeutic agent can be a cytokine such as G-CSF (granulocyte colony stimulating factor). In another aspect, a compound of the present invention, or a pharmaceutically acceptable salt, prodrug, metabolite, analog or derivative thereof, may be administered in combination with radiation therapy. Radiation therapy can also be administered in combination with a compound of the present invention and another chemotherapeutic agent described herein as part of a multiple agent therapy. In yet another aspect, a compound of the present invention, or a pharmaceutically acceptable salt, prodrug, metabolite, analog or derivative thereof, may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™) or CMFP (cyclophosphamide, methotrexate, 5-fluorouracil and prednisone).

In preferred embodiments, a compound of the present invention, or a pharmaceutically acceptable salt, prodrug, metabolite, polymorph or solvate thereof, may be administered with an inhibitor of an enzyme, such as a receptor or non-receptor kinase. Receptor and non-receptor kinases of the invention are, for example, tyrosine kinases or serine/threonine kinases. Kinase inhibitors of the invention are small molecules, polynucleic acids, polypeptides, or antibodies.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevic (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER2/Erb2), Panitumumab/Vectibix (targets EGFR), Vandetinib (targets RET/VEGFR), E7080 (multiple targets including RET and VEGFR), Herceptin (targets HER2/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-β, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER3), and AP24534 (multiple targets including Flt3).

Exemplary serine/threonine kinase inhibitors include, but are not limited to, Rapamune (targets mTOR/FRAP1), Deforolimus (targets mTOR), Certican/Everolimus (targets mTOR/FRAP1), AP23573 (targets mTOR/FRAP1), Eril/Fasudil hydrochloride (targets RHO), Flavopiridol (targets CDK), Seliciclib/CYC202/Roscovitrine (targets CDK), SNS-032/BMS-387032 (targets CDK), Ruboxistaurin (targets PKC), Pkc412 (targets PKC), Bryostatin (targets PKC), KAI-9803 (targets PKC), SF1126 (targets PI3K), VX-680 (targets Aurora kinase), Azd1152 (targets Aurora kinase), Arry-142886/AZD-6244 (targets MAP/MEK), SCIO-469 (targets MAP/MEK), GW681323 (targets MAP/MEK), CC-401 (targets JNK), CEP-1347 (targets JNK), and PD 332991 (targets CDK).

The present invention also provides pharmaceutical compositions comprising a candidate compound of the present invention in combination with at least one pharmaceutically acceptable excipient or carrier.

A “pharmaceutical composition” is a formulation containing the compounds of the present invention in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the invention can be administered to a subject in many of the well-known methods currently used for chemotherapeutic treatment. For example, for treatment of cancers, a compound of the invention may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

Preferably, the therapeutically effective amount is the amount of the candidate compound to induce or increase the phosphorylation status/level of NF45 in a cell. More preferably, the amount of the candidate compound to induce or increase the phosphorylation status/level of NF45 on the Threonine at amino acid residue 388 of SEQ ID NO: 1

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The compounds of the present invention are capable of further forming salts. All of these forms are also contemplated within the scope of the claimed invention.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The compounds of the present invention can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

The compounds of the present invention can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a subject. Prodrugs in the present invention are prepared by modifying functional groups present in the compound in such a way that the modifications are cleaved, either in routine manipulation or in vivo, to the parent compound. Prodrugs include compounds of the present invention wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the invention, and the like, See Bundegaard, H., Design of Prodrugs, p 1-92, Elesevier, New York-Oxford (1985).

The compounds, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Techniques for formulation and administration of the disclosed compounds of the invention can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### EXAMPLES

### Example 1

### Phosphorylated NF45 (T388) Antibodies and Identification of Phosphorylated NF45 (T388) as a Pharmacodynamic Biomarker

Cell culture and chemicals: MIA PaCa-2 cells were maintained in DMEM medium supplemented with 10% FBS. β-Lapachone and (S)-1′-(3-(4-(tert-butyl)phenoxy)-2-hydroxypropyl)-2H-spiro[naphtho[1,2-b][1,4]oxathiine-3,4′-piperidine]-5,6-dione were synthesized by ArQule, Inc. Doxorubicin, cisplatin, camptohethcin and NU7026 (DNA-PK inhibitor) were purchased from Sigma. KU55933 (ATM inhibitor) was purchased from EMD Chemicals.

Identification of NF45: MIA PaCa-2 cells were treated with or without β-Lapachone (4 mM) for 8 hrs and Western blot analysis was performed using anti-threonine antibody (#9381, Cell Signaling Technology). A replicate gel was stained with Colloidal Blue or silver stain (Invitrogen). The 45 kD band corresponding to immunoreactivity for phospho threonine was excised and sent to Beth Israel Deaconess Medical Center Genomics Center Proteome Core for protein analysis.

Antibody generation: Anti-pNF45 (T388) antibody was generated by Covance (Norwood, Mass.). Briefly, a peptide corresponding to amino acids 383 to 390 (EESME-T-QE) (SEQ ID NO: 2) of NF45 was phosphorylated at threonine 388 in vitro and then conjugated with KHL. Polyclonal antibody was produced from rabbit and passed through affinity column with unphosphorylated peptide and then purified through the column with phosphorylated peptide. Two batches of antibody were obtained: #438 and #439. All the experiments were carried out using #438. Monoclonal antibody are generated by utilizing SEQ ID NO:2 and the hybridoma method known in the art and described herein.

Immunoprecipitation and Western Blot Analysis: Cell lysate was used for immunoprecipitation using anti-NF90 antibody (#612154, BD Biosciences) or IgG as negative control. Samples then were separated into two equal potions: one blotted with anti-pNF45(T388)(#438) and another for anti-NF45 (K-22) (sc-133817, Santa Cruz Biotechnology). Other antibodies used in this study are pATM(S1981)(# 200-301-400, Rockland), DNA-PKcs (MA-370-P0, Thermo Scientific), ATM(2C1) (GTX70103, GeneTex), DNA-PK (S2506) (ab18192, Abcam), DNA-PK (T2609) (ab18356, Abcam), pCHK2(T68) (#2661, CST), b-actin (#5441, Sigma), g-H2AX (A300-081A, Bethyl).

Knockdown of NF45: Cells were transfected with NF45 On-target plus SMART pool (L-017599-00-0005, Thermo Fisher) or On-Target plus Control siRNA (200 nM, #D-001810-0X, Thermo Fisher) using Oligofectamine (#12252-011, Invitrogen). Cells were used for further examination after 48 hr post transfection.

Immunofluorescent Staining: Cells were seeded in 8-well chamber slides and treated with or without β-Lapachone for various time periods and then fixed with 4% paraformaldhyde. pNF45 was detected using anti-pNF45(T388) antibody, and NF45 using anti-NF45 (K-22) antibody. Slides were mounted using Vectashield Mounting Medium with DAPI (#H-1200, Vector Laboratories) and images were obtained using Zeiss fluorescence microscope.

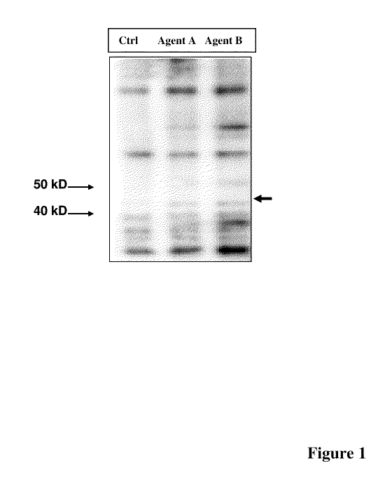

Identification of NF45: MIA PaCa-2 cells were treated with or without β-Lapachone (4 μM) (“Agent A”) or (S)-1′-(3-(4-(tert-butyl)phenoxy)-2-hydroxypropyl)-2H-spiro[naphtho[1,2-b][1,4]oxathiine-3,4′-piperidine]-5,6-dione (4 μM) (“Agent B”) for 8 hrs and the cell lysate was subjected to western blot analysis for phospho-threonine. FIG. 1, Panel A, shows highly phosphorylated 45 kD protein band (arrow), which was excised for protein identification.

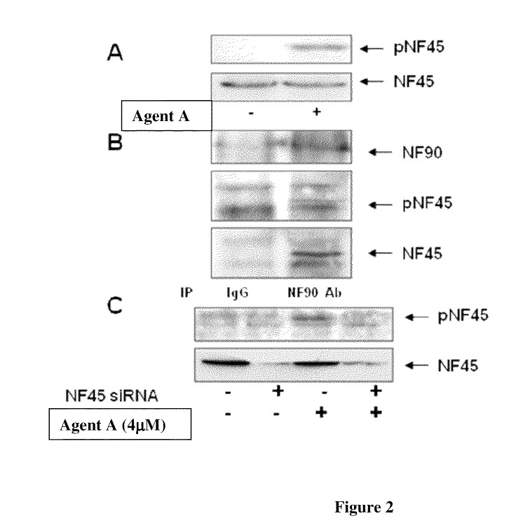

Threonine Phosphorylation of pNF45 in MIA PaCa-2 Cells is Responsive to β-Lapachone: MIA PaCa-2 cells were treated with or without β-Lapachone (4 μM) (“Agent A”) for 8 hrs and cell lysate was subjected to western blot analysis for the detection of pNF45(T388). FIG. 2, Panel A, shows that β-Lapachone induced pNF45 (T388). FIG. 2, Panel B, shows an immunoprecipitation assay performed using anti-NF90 antibody. Samples were then separated into two portions: one for detection of pNF45 (T388) and another for detection of total NF45. C. Cells were transfected with NF45 siRNA or control siRNA and then treated with or without β-Lapachone (4 μM) for 8 hrs. FIG. 2, Panel C, shows that knockdown of NF45 abolished phosphorylation of NF45. Taken together, these results indicate that β-Lapachone is able to induce phosphorylation of NF45 at threonine 388, which is recognized by anti-pNF45 (T388) antibody.

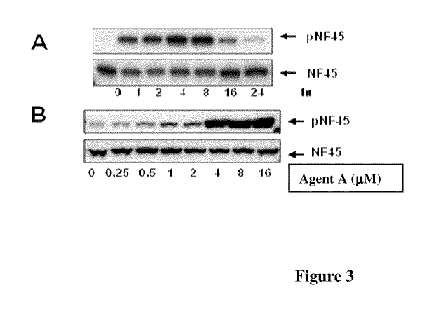

Time- and concentration-dependent pNF45 (T388) in response to β-Lapachone in MIA PaCa-2 cells: MIA PaCa-2 cells were treated with β-Lapachone (4 μM) (“Agent A”) for various times (A), or for 2 hrs at various concentrations of β-Lapachone (B). Western blot analysis was performed for the detection of pNF45 (T388) and NF45. FIG. 3 shows that β-Lapachone induces pNF45 (T388) in a time- and concentration-dependent manner. Early induction of pNF45 (T388) was observed within 1 hr and reached peak level between 4 to 8 hrs.

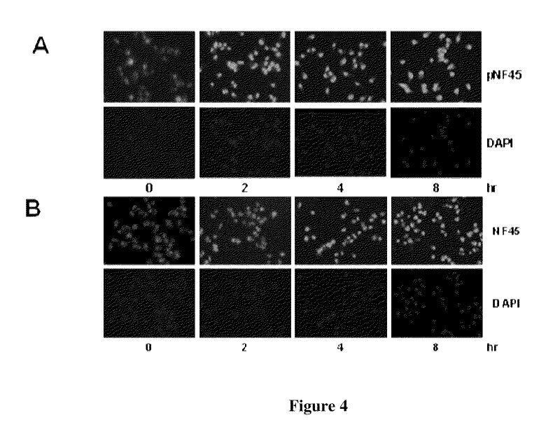

Immunofluorescence staining pNF45 (T388) in response to β-Lapachone: MIA PaCa-2 cells were plated in 8-well chamber slides and treated with β-Lapachone (4 μM) (“Agent A”) for various times. Cells were fixed and stained for either pNF45 (A) or NF45 (B). FIG. 4 shows β-Lapachone induction of pNF45 (T388) within 2 hrs and pNF45 (T388) is primarily located in nuclei.

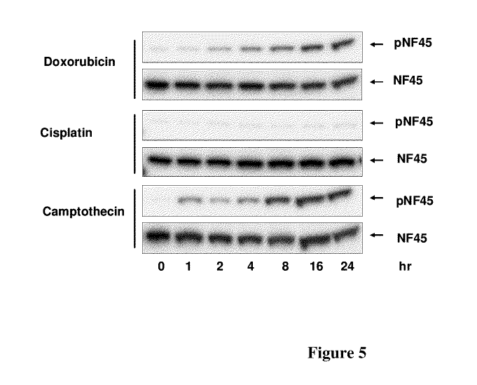

Effect of DNA damage agents on phosphorylation of NF45: To determine whether other DNA damage agents are able to regulate NF45, we treated MIA Paca-2 cells with or without doxorubicin (1 μM), cisplatin (10 μM) or camptothecin (10 μM) for various times. pNF45 (T388) was examined by western blot analysis. The results in FIG. 5 show that doxorubicin and camptothecin but not cisplatin induced NF45 phosphorylation, which steadily increased up to 24 hrs.

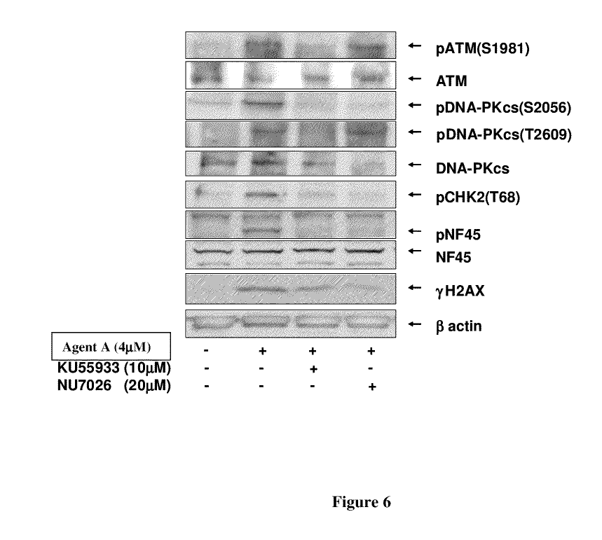

β-Lapachone-induced phosphorylation of NF45 is ATM- and DNA-PK-dependent: MIA PaCa-2 cells were pretreated with or without ATM inhibitor KU55933 (10 μM), or DNA-PK inhibitor NU7026 (20 μM), for 1 hr and then treated with β-Lapachone (4 μM) (“Agent A”) for 8 hrs in the presence of inhibitors. Western blot analysis was performed using specific antibodies. The results in FIG. 6 show that phosphorylation of NF45 induced by β-Lapachone is ATM and DNA-PK dependent since β-Lapachone induced DNA damage response in cells.

These results of Example 1 show that quinones, such as β-Lapachone, induce NF45 phosphorylation at threonine 388 in a time- and concentration-dependent manner in MIA PaCa-2 cells Immunofluorescence staining shows that phosphorylation of NF45 induced by quinines, such as β-Lapachone, is primarily located in nuclei, suggesting that pNF45 (T388) could be a PD biomarker for quinone therapy (e.g., β-Lapachone therapy). Doxorubicin and camptothecin but not cisplatin increased pNF45 (T388) in a time-dependent fashion. Unlike β-Lapachone, those two DNA damaging agents induced pNF45 (T388) strongly at a later time. Thus, β-Lapachone may have different mechanism in regulating pNF45 from doxorubicin and camptothecin. Induction of pNF45 (T388) by β-Lapachone coincides with activation of ATM and DNA-PK Inhibition of ATM or DNA-PK using specific inhibitors suppresses pNF45(T388) induced by β-Lapachone, suggesting that phosphorylation of NF45 is ATM and DNA-PK dependent.

We claim:

1. An isolated antibody or antigen-binding portion thereof that specifically binds a protein comprising the amino acid sequence of SEQ ID NO: 1 phosphorylated at one or more serine, threonine, or tyrosine residues.

2. The isolated antibody of claim 1, wherein the antibody specifically binds the protein of SEQ ID NO: 1 phosphorylated at one or more threonine residues.

3. The isolated antibody of claim 1, wherein the antibody specifically binds the protein of SEQ ID NO: 1 phosphorylated at threonine residue T 388.

4. An isolated peptide comprising the amino acid sequence of SEQ ID NO: 1 phosphorylated at one or more serine, threonine, or tyrosine residues.

5. The isolated peptide of claim 4, wherein the peptide is phosphorylated at threonine residue T 388.

6. A method comprising (a) contacting a biological sample with the isolated antibody of claim 1; and, (b) assessing a complex formed between peptide comprising the amino acid sequence of SEQ ID NO:1 phosphorylated at one or more serine, threonine, or tyrosine residues and said antibody to determine the presence, absence, or amount of said peptide in said biological sample.

7. The isolated peptide of claim 6, wherein the peptide is phosphorylated at threonine residue T 388.

8. The method of claim 6, wherein the antibody specifically binds to the peptide phosphorylated at threonine residue T388.

9. A method comprising (a) contacting a biological sample with a candidate compound; (b) determining the presence, absence, or amount of a peptide comprising the amino acid sequence of SEQ ID NO:1 phosphorylated at one or more serine, threonine, or tyrosine residues in said biological sample following contact with the candidate compound; and, (c) comparing the presence, absence, or amount of said peptide in said biological sample with the presence, absence, or amount of said peptide determined in a biological sample not contacted with the candidate compound.

10. A method comprising (a) exposing a subject to a candidate compound; (b) obtaining a biological sample from said subject following said exposure; (c) determining the presence, absence, or amount of a peptide comprising the amino acid sequence of SEQ ID NO:1 phosphorylated at one or more serine, threonine, or tyrosine residues in said biological sample; and, (d) comparing the presence, absence, or amount of said peptide in said biological sample with the presence, absence, or amount of said peptide determined in a biological sample obtained from a subject not exposed to the candidate compound.

11. The method of claim 9 or 10, wherein said determining comprises contacting said biological sample with an isolated antibody or antigen-binding portion thereof that specifically binds to said peptide comprising the amino acid sequence of SEQ ID NO:1 phosphorylated at one or more serine, threonine, or tyrosine residues and assessing a complex formed between said peptide and said antibody.

12. The method of claim 9 or 10, wherein the peptide is phosphorylated at threonine residue T388.

13. The method of claim 12, wherein the antibody specifically binds to the peptide phosphorylated at threonine residue T388.

14. The method of claim 9 or 10, further comprising identifying a candidate compound capable of inducing or increasing said peptide.

15. The method of claim 9 or 10, wherein the candidate compound is a napthoquinone compound or a topoisomerase inhibitor.

16. The method of claim 10, further comprising identifying a subject having an induction or increase in said peptide following exposure to said candidate compound and administering a chemotherapeutic agent or radiation therapy to said subject.

17. The method of claim 16, wherein said subject has a proliferation disorder.

18. The method of claim 17, wherein said proliferation disorder is cancer.

19. The method of claim 17, wherein said administration of the chemotherapeutic agent or radiation therapy treats the proliferation disorder in said subject.
